(19) United States
(12) Patent Application Publication
(10) Pub. No.: US 2007/0142311 A1
(43) Pub. Date: Jun. 21, 2007

(54) Diagnosis of hyperinsulinemia and type II diabetes and protection against same

(76) Inventors: John J. Kopchick, Athens, OH (US); Bruce Kedler, Athens, OH (US); Keith S Boyce, Wexford, OH (US); Andres Kriete, Pittsburgh, PA (US)

Correspondence Address: BROWDY AND NEIMARK, P.L.L.C.;624 NINTH STREET, NW, SUITE 300, WASHINGTON, DC, 20001-5303, US

(21) Appl. No.: 10/552,443

(22) PCT Filed: Apr. 2, 2004
(86) PCT No.: PCT/US04/10191

§ 371 Date: Aug. 16, 2006

Related U.S. Application Data
(60) Provisional application No. 60/460,415, filed on Apr. 7, 2003.
(60) Provisional application No. 60/506,716, filed on Sep. 30, 2003.

(51) Int. Cl. A61K 31/70; A01N 43/04

(52) U.S. Cl. 514/44

(57) ABSTRACT

Mouse genes differentially expressed in comparisons of normal vs. hyperinsulinemic, hyperinsulinemic vs. type 2 diabetic, and normal vs. type 2 diabetic liver by gene chip analysis have been identified, as have corresponding human genes and proteins. The human molecules, or antagonists thereof, may be used for protection against hyperinsulinemia or type 2 diabetes, or their sequelae.

This application claims the benefit under 35 USC 119(e) of prior U.S. provisional applications 60/460,415, filed Apr. 7, 2003 (KOPCHICK6-USA), and 60/506,716, filed Sep. 30, 2003 (KOPCHICK6.1-USA), both of which are hereby incorporated by reference in their entirety.

### CROSS-REFERENCE TO RELATED APPLICATIONS

The instant application adds 6 month expression data to the disclosure of U.S. Prov. Appl. 60/460,415, filed Apr. 7, 2003 (KOPCHICK6-USA).

In U.S. Provisional Appl. Ser. No. 60/458,398 (our docket Kelder1-USA), filed Mar. 31, 2003, we describe the identification of genes differentially expressed in normal vs. hyperinsulinemic, hyperinsulinemic vs. type II diabetic, or normal vs. type II diabetic mouse liver. Forward- and reverse-substracted cDNA libraries were prepared, clones were isolated, and differentially expressed cDNA inserts were sequenced and compared with sequences in publicly available sequence databases. The corresponding mouse and human genes and proteins were identified. Favorable genes/proteins so identified included (1) NP_{—}000767: cytochrome P450, subfamily IIIA (niphedipine oxidase), polypeptide 3; (2) AAG31034: SYT/SSX4 fusion protein; and (3) NP_{—}003158: sulfotransferase family, cytosolic, 2A, dehydroepiandrosterone (DHEA)-preferring, member 1; sulfotransferase family 2A, dehydroepiandrosterone (DHEA)-preferring, member 1. Unfavorable proteins included (4) NP_{—}004884: H2A histone family, member Y isoform 2; histone macroH2A1.2; histone macroH2A1.1; (5) AAH37738: Unknown (protein for MGC:33851); (6) NP_{—}068839: integral membrane protein 2B ; (7) CAA28659: S-protein ; and (8) AAA51560: alpha-1-antichymotrypsin precursor. Mixed proteins included (9) NP_{—}000769: cytochrome P450, subfamily IVA, polypeptide 11; fatty acid omega-hydroxylase; P450HL-omega; alkane-1monooxygenase; lauric acid omega-hydroxylase; (10) NP_{—}006206:serine (or cysteine) proteinase inhibitor, clade A ; (11) NP_{—}004489: one cut domain, family member 1; hepatocyte nuclear factor 6, alpha; and (12) NP_{—}775491: liver-specific uridine phosphorylase. Gene chip technology was not used. Two of the genes (NM_{—}007818 and NM_{—}007818) were also identified in the present case.

The use of differential hybridization to identify genes and proteins is also described in our Ser. No. PCT/US00/12145 (Kopchick 3A-PCT), Ser. No. PCT/US00/12366 (Kopchick4A-PCT), and Ser. No. 60/400,052 (Kopchick5). All of the above applications are incorporated by reference in their entirety.

### BACKGROUND OF THE INVENTION

1. Field of the Invention

The invention relates to various nucleic acid molecules and proteins, and their use in (1) diagnosing hyperinsulinemia and type II diabetes, or conditions associated with their development, and (2) protecting mammals (including humans) against them.

2. Description of the Background Art

Diabetes

Diabetes mellitus is a pleiotropic disease of great complexity. The two major types have been termed type I or insulin-dependent diabetes mellitus (IDDM) and type II or non-insulin-dependent diabetes mellitus (NIDDM). Type II diabetes is the predominant form found in the Western world; fewer than 8% of diabetic Americans have the type I disease.

Type I diabetics are often characterized by their low or absent levels of circulating endogenous insulin, i.e., hypoinsulinemia (1). Islet cell antibodies causing damage to the pancreas are frequently present at diagnosis. Injection of exogenous insulin is required to prevent ketosis and sustain life.

Early Type II diabetics are often characterized by hyperinsulinemia and resistance to insulin. Late Type II diabetics may be normoinsulinemic or hypoinsulinemic. Type II diabetics are usually not insulin dependent or prone to ketosis under normal circumstances.

Type II Diabetes

Type II diabetes (formerly known as non-insulin dependent diabetes, NIDDM) is the most common form of elevated blood glucose (hyperglycemia). Type II diabetes is a metabolic disorder that affects approximately 17 million Americans. It is estimated that another 10 million individuals are “prone” to becoming diabetic. These vulnerable individuals can become resistant to insulin, a pancreatic hormone that signals glucose (blood sugar) uptake by fat and muscle. In order to maintain normal glucose levels, the islet cells of the pancreas produce more insulin, resulting in a condition called hyperinsulinemia. When the pancreas can no longer produce enough insulin to compensate for the insulin resistance, and thereby maintain normal glucose levels, Type II diabetes (hyperglycemia) results.

Complications of diabetes (end organ damage) include retinopathy, neuropathy, and nephropathy (traditionally designated as microvascular complications) as well as atherosclerosis (a macrovascular complication).

Early stages of hyperglycemia can usually be controlled by an alteration in diet and increasing the amount of exercise, but drug treatment, including insulin, may be required. It has been shown that meticulous blood glucose control can often slow down or halt the progression of diabetic complications if caught early enough (1). However, tight metabolic control is extremely difficult to achieve.

Little is known about the disease progression from the normoinsulinemic state to the hyperinsulinemic state, and from the hyperinsulinemic state to the Type II diabetic state.

As stated above, type II diabetes is a metabolic disorder that is characterized by insulin resistance and impaired glucose-stimulated insulin secretion (2,3,4). However, Type II diabetes and atherosclerotic disease are viewed as consequences of having the insulin resistance syndrome (IRS) for many years (5). The current theory of the pathogenesis of Type II diabetes is often referred to as the “insulin resistance/islet cell exhaustion” theory. According to this theory, a condition causing insulin resistance compels the pancreatic islet cells to hypersecrete insulin in order to maintain glucose homeostasis. However, after many years of hypersecretion, the islet cells eventually fail and the symptoms of clinical diabetes are manifested. Therefore, this theory implies that, at some point, peripheral hyperinsulinemia will be an antecedent of Type II diabetes. Peripheral hyperinsulinemia can be viewed as the difference between what is produced by the β cell minus that which is taken up by the liver. Therefore, peripheral hyperinsulinemia can be caused by increased β cell production, decreased hepatic uptake or some combination of both. It is also important to note that it is not possible to determine the origin of insulin resistance once it is established since the onset of peripheral hyperinsulinemia leads to a condition of global insulin resistance.

Multiple environmental and genetic factors are involved in the development of insulin resistance, hyperinsulinemia and type II diabetes. An important risk factor for the development of insulin resistance, hyperinsulinemia and type II diabetes is obesity, particularly visceral obesity (6,7,8). Type II diabetes exists world-wide, but in developed societies, the prevalence has risen as the average age of the population increases and the average individual becomes more obese.

Obesity is a serious and growing problem in the United States. Obesity-related health risks include high blood pressure, hardening of the arteries, cardiovascular disease, and Type II diabetes (also known as non-insulin-dependent diabetes mellitus, Type II diabetes)(9,10,11). Recent studies show that 85% of the individuals with Type II diabetes are obese (12).

Growth Hormone

Growth hormone has many roles, ranging from regulation of protein, fat and carbohydrate metabolism to growth promotion. GH is produced in the somatrophic cells of the anterior pituitary and exerts its effects either through the GH-induced action of IGF-I, in the case of growth promotion, or by direct interaction with the GHR on target cells including liver, muscle, adipose, and kidney cells. Hyposecretion of GH during development leads to dwarfism, and hypersecretion before puberty leads to gigantism. In adults, hypersecretion of GH results in acromegaly, a clinical condition characterized by enlarged facial bones, hands, feet, fatigue and an increase in weight. Of those individuals with acromegaly, 25% develop type II diabetes. This may be due to insulin resistance caused by the high circulating levels of GH leading to high circulating levels of insulin (Kopchick et al., Annual Rev. Nutrition 1999. 19:437-61).

A further mode of GH action may be through the transcriptional regulation of a number of genes contributing to the physiological effects of GH.

Transgenic Mice

McGrane, et al., J. Biol. Chem. 263:11443-51 (1988) and Chen, et al., J. Biol. Chem., 269:15892-7 (1994) describe the genetic engineering of mice to express bovine growth hormone (bGH) or human growth hormone (hGH), respectively. These mice exhibited an enhanced growth phenotype. They also developed kidney lesions similar to those seen in diabetic glomerulosclerosis, see Yang, et al., Lab. Invest., 68:62-70 (1993). Ogueta, et al., J. Endocrinol., 165: 321-8 (2000) reported that transgenic mice expressing bovine GH develop arthritic disorder and self-antibodies.

Growth hormone genes and the proteins encoded by them can be converted into growth hormone antagonists by mutation, see Kopchick U.S. Pat. No. 5,350,836. Transgenic mice have been made that express the GH antagonists bGH-G119R or hGH G120R, and which exhibit a dwarf phenotype. Chen, et al., J. Biol. Chem., 263:15892-7 (1994); Chen, et al., Mol. Endocrinol, 5:1845-52 (1991); Chen, et al., Proc. Nat. Acad. Sci. USA 87:5061-5 (1990). These mice did not develop kidney lesions. See Yang (1993), supra.

Chen, et al., Endocrinol, 136:660-7 (1995) compared the effect of streptozotocin treatment in normal nontransgenic mice, and in mice transgenic for (1) a GH receptor antagonist, the G119R mutant of bovine growth hormone or (2) the E117L-mutant of bGH. (According to Chen's ref. 24, these large GH transgenic streptozotocin-treated mice constitute an animal model for diabetes.) Glomerulosclerosis was seen in diabetic (STZ-treated) nontransgenic mice and in diabetic bGH-E117L mice, but not in diabetic bGH-G119R (GH antagonist) mice.

Two of the proteins which mediate growth hormone activity are the growth hormone receptor and the growth hormone binding protein, encoded by the same gene in mice (GHR/BP). It is possible to genetically engineer mice so that the gene encoding these proteins is disrupted (“knocked-out”; inactivated), see Zhou, et al., Proc. Nat. Acad. Sci. (USA), 94:13215-20 (1997). Zhou, et al. inactivated the GHR/BP gene by replacing the 3′ portion of exon 4 (which encodes a portion of the GH binding domains) and the 5′ region of intron 4 with a neomycin gene cassette. The modified gene was introduced into the target mice by homologous recombination. Like mice expressing a GH antagonist, homozygous GHR/BP-KO mice exhibit a dwarf phenotype. GHR/BP-KO mice, made diabetic by streptozotocin treatment, are protected from the development of diabetes-associated nephropathy. Bellush, et al., Endocrinol., 141:163-8 (2000).

Differential/Subtractive Hybridization

Zhang, et al., Kidney International, 56:549-558 (1999) identified genes up-regulated in 5/6 nephrectomized (subtotal renal ablation) mouse kidney by a PCR-based subtraction method. Ten known and nine novel genes were identified. The ultimate goal was to identify genes involved in glomerular hyperfiltration and hypertrophy.

Melia, et al., Endocrinol., 139:688-95 (1998) applied subtractive hybridization methods for the identification of androgen-regulated genes in mouse kidney. The treatment mice were dosed with dihydrotestosterone, an androgen. Kidney androgen-regulated protein gene was used as a positive control, as it is known to be up-regulated by DHT.

See also Holland, et al., Abstract 607, “Identification of Genes Possibly Involved in Nephropathy of Bovine Growth Hormone Transgenic Mice” (Endocrine Society Meeting, Jun. 22, 2000) and Coschigano, et al., Abstract 333, “Identification of Genes Potentially Involved in Kidney Protection During Diabetes” (Endocrine Society Meeting, Jun. 22, 2000).

The following differential hybridization articles may also be of interest:

Wada, et al., “Gene expression profile in streptozotocin-induced diabetic mice kidneys undergoing glomerulosclerosis”, Kidney Int, 59:1363-73 (2001);

Song, et al., “Cloning of a novel gene in the human kidney homologous to rat muncl3S: its potential role in diabetic nephropathy”, Kidney Int., 53:1689-95 (1998);

Page, et al., “Isolation of diabetes-associated kidney genes using differential display”, Biochem. Biophys. Res. Comm., 232:49-53 (1997).

Peradi, “Subtractive hybridization claims: An efficient technique to detect overexpressed mRNAs in diabetic nephropathy,” Kidney Int. 53:926-31 (1998).

Condorelli, EMBO J., 17:3858-66 (1998).

See also WO00/66784 (differential hybridization screening for brown adipose tissue); PCT/US00/2366, filed May 5, 2000 (differential hybridization screening for liver).

Identification of Genes Involved in Hyperinsulinemia and Type II Diabetes

High-fat diets have been shown to induce both obesity and Type II diabetes in laboratory animals (13). Surwit and colleagues demonstrated that male C57BL/6J mice are extremely sensitive to the diabetogenic effects of a high-fat diet when initiated at weaning. At six months of age, high-fat fed animals had significantly elevated fasting blood-glucose and insulin levels and also demonstrated a decrease in insulin sensitivity (14). Ahren and colleagues (15) reported evidence of insulin resistance as well as diminished glucose-stimulated insulin release, after feeding with a high-fat diet for 12 weeks. These mice also showed elevated levels of total cholesterol, triglycerides, and free fatty acids, another hallmark of Type II diabetes.

Our attention recently has focused on the generation of liver mRNA expression profiles and the identification of genes involved in the genesis of the obesity-induced hyperinsulinemia and type-II diabetes. To date, no one has attempted to study the actual progression from the normal condition to that of hyperinsulinemia or from hyperinsulinemia to Type II diabetes in an attempt to identify genes that are up-regulated or down-regulated as the disease progresses.

In previous studies aimed at identifying genes involved in diabetes-induced glomerulosclerosis, differential display and traditional subtractive hybridization techniques were used (16-20). While effective for the identification of a few genes (e.g. hmunc13, PED/PEA-15, lactate dehydrogenase, amiloride sensitive sodium channel, ubiquitin-like protein, mdr 1, and a-amyloid protein precursor as well as a few novel genes), these techniques can be quite labor intensive. The PCR-based method of subtractive hybridization requires less starting material, and allows the simultaneous isolation of all differentially expressed cDNAs into two groups (up-regulated and down-regulated).

However, the PCR-based method of subtractive hybridization is also quite labor-intensive, produced large numbers of false positive candidates and ultimately resulted in the identification of a relatively limited number of differentially expressed genes. (see Kelder1-USA application).

In order to expand the number of genes that can be analyzed simultaneously, several groups have begun to utilize DNA microarray analysis to measure differences in gene expression between normal and diseased states. However, these experiments have been limited in regards to the number of experimental conditions analyzed. DNA microarray analysis has been performed on normal, obese and diabetic mice (21). Also, the obesity and diabetes in the mouse models examined were caused by a specific endogenous genetic mutation (22). The differentially expressed genes in the above models may be very different from genes differentially expressed due to diet-induced obesity and Type-II diabetes.

### SUMMARY OF THE INVENTION

Differential hybridization techniques have been used to identify mouse genes that are differentially expressed in mice, depending upon their development of hyperinsulinemia or type II diabetes.

In essence, complementary RNA derived from normal mice, or mouse models of hyperinsulinemia or type II diabetes, was screened for hybridization with oligonucleotide probes each specific to a particular mouse gene, each gene in turn representative of a particular mouse gene cluster (Unigene). Mouse genes which were differentially expressed (normal vs. hyperinsulinemic, hyperinsulinemic vs. diabetic, or normal vs. diabetic), as measured by different levels of hybridization of the respective cRNA samples with the particular probe corresponding to that mouse gene) were identified. Related human genes and proteins were identified by sequence comparisons to the mouse gene or protein.

After identifying related human genes and proteins, one may formulate agents useful in screening humans at risk for progression toward hyperinsulinemia or toward type II diabetes.

Since the progression is from normal to hyperinsulinemic, and thence from hyperinsulinemic to type II diabetic, one may define mammalian subjects as being more favored or less favored, with normal subjects being more favored than hyperinsulinemic subjects, and hyperinsulinemic subjects being more favored than type II diabetic subjects. The subjects' state may then be correlated with their gene expression activity.

Thus, “favorable” human genes/proteins are defined as those corresponding to mouse genes which were less strongly expressed in mouse hyperinsulinemic liver than in control liver, or less strongly expressed in mouse type II diabetic liver than in hyperinsulinemic liver. (The control liver is the liver of a mouse which is normal vis-a-vis fasting insulin and fasting glucose levels. The term “normal”, as used herein, means normal relative to those parameters, and does not necessitate that the mouse be normal in every respect.) Likewise, one may define “unfavorable” human genes/proteins as those corresponding to mouse genes which were more strongly expressed in mouse hyperinsulinemic liver than in control liver, or more strongly expressed in mouse type II diabetic liver than in hyperinsulinemic liver.

As used herein, the term “corresponding” does not mean identical, but rather implies the existence of a statistically significant sequence similarity, such as one sufficient to qualify the human protein or gene as a homologus protein or DNA as defined below. The greater the degree of relationship as thus defined (i.e., by the statistical significance of each alignment used to connect the mouse cDNA to the human protein or gene, measured by an E value), the more close the correspondence. The connection may be direct (mouse gene to human protein) or indirect (e.g., mouse gene to human gene, human gene to human protein). By “mouse gene”, we mean the mouse gene from which the gene chip DNA in question was derived.

In general, the human genes/proteins which most closely correspond, directly or indirectly, to the mouse genes are preferred, such as the one(s) with the highest, top two highest, top three highest, top four highest, top five highest, and top ten highest E values for the final alignment in the connection process. The human genes/proteins deemed to correspond to our mouse cDNA clones are identified in the Master Tables.

A human gene/protein corresponding to a mouse cDNA which was more strongly expressed in hyperinsulinemic liver than in either normal or type II diabetic liver (i.e., C<HI, HI>D) will be deemed both “unfavorable”, by virtue of the control:hyperinsulinemic comparison, and “favorable”, by virtue of the hyperinsulinemic:diabetic comparison. This is one of several possible “mixed” expression patterns.

Thus, we can subdivide the “favorables” into wholly and partially favorables. Likewise, we can subdivide the unfavorables into wholly and partially unfavorables. The genes/proteins with “mixed” expression patterns are, by definition, both partially favorable and partially unfavorable. In general, use of the wholly favorable or wholly unfavorable genes/proteins is preferred to use of the partially favorable or partially unfavorable ones.

Agents which bind the “favorable” and “unfavorable” nucleic acids (e.g., the agent is a substantially complementary nucleic acid hybridization probe), or the corresponding proteins (e.g., an antibody vs. the protein) may be used to evaluate whether a human subject is at increased or decreased risk for progression toward type II diabetes. A subject with one or more elevated “unfavorable” and/or one or more depressed “favorable” genes/proteins is at increased risk, and one with one or more elevated “favorable” and/or one or more depressed “unfavorable” genes/proteins is at decreased risk. One may further take into account whether the subject is normoinsulinemic or hyperinsulinemic at the time of the assay. If the subject is non-diabetic and normoinsulinemic, we are especially interested in the “favorable” and “unfavorable” genes/proteins corresponding to mouse genes differentially expressed in hyperinsulinemic vs. normal livers. If the subject is already hyperinsulinemic, yet non-diabetic, we are especially interested in the “favorable” and “unfavorable” genes/proteins corresponding to mouse genes differentially expressed in type II diabetic vs. hyperinsulinemic livers.

The assay may be used as a preliminary screening assay to select subjects for further analysis, or as a formal diagnostic assay.

The identification of the related genes and proteins may also be useful in protecting humans against these disorders.

Thus, Applicants contemplate:

(1) use of the “favorable” mouse DNAs of the Master Tables (below) to isolate or identify related human DNAs;

(2) use of human DNAs, related to favorable mouse DNAs, to express the corresponding human proteins;

(3) use of the corresponding human proteins (and mouse proteins, if biologically active in humans), to protect against the disorder(s);

(4) use of the corresponding mouse or human proteins, or nucleic acid probes derived from the mouse or human genes, in diagnostic agents, in assays to measure progression toward hyperinsulinemia or type II diabetes, or protection against the disorder(s), or to estimate related end organ damage such as kidney damage; and

(5) use of the corresponding human or mose genes therapeutically in gene therapy, to protect against the disorder(s).

Moreover Applicants contemplate:

(1) use of the “unfavorable” mouse DNAs of the Master Tables to isolate or identify related human DNAs;

(2) use of the complement to the “unfavorable” mouse DNAs or related human DNAs, as antisense molecules to inhibit expression of the related human DNAs;

(3) use of the mouse or human DNAs to express the corresponding mouse or human proteins;

(4) use of the corresponding mouse or human proteins, in diagnostic agents, to measure progression toward hyperinsulinemia or type II diabetes, or protection against the disorder(s), or to estimate related end organ damage such as kidney damage;

(5) use of the corresponding mouse or human proteins in assays to determine whether a substance binds to (and hence may neutralize) the protein; and

(6) use of the neutralizing substance to protect against the disorder(s).

The related human DNAs may be identified by comparing the mouse sequence (or its AA translation product) to known human DNAs (and their AA translation products). If this is unsuccessful, human cDNA or genomic DNA libraries may be screened using the mouse DNA as a probe.

### DETAILED DESCRIPTION OF THE PREFERRED EMBODIMENTS OF THE INVENTION

Subjects

A mouse is considered to be a diabetic subject if, regardless of its fasting plasma insulin level, it has a fasting plasma glucose level of at least 190 mg/dL. A mouse is considered to be a hyperinsulinemic subject if its fasting plasma insulin level is at least 0.67 ng/mL and it does not qualify as a diabetic subject. A mouse is considered to be “normal” if it is neither diabetic nor hyperinsulinemic. Thus, normality is defined in a very limited manner.

A mouse is considered “obese” if its weight is at least 15% in excess of the mean weight for mice of its age and sex. A mouse which does not satisfy this standard may be characterized as “non-obese”, the term “normal” being reserved for use in reference to glucose and insulin levels as previously described.

A human is considered a diabetic subject if, regardless of his or her fasting plasma insulin level, the fasting plasma glucose level is at least 126 mg/dL. A human is considered a hyperinsulinemic subject if the fasting plasma insulin level is more than 26 micro International Units/mL (it is believed that this is equivalent to 1.08 ng/mL), and does not qualify as a diabetic subject. A human is considered to be “normal” if it is neither diabetic nor hyperinsulinemic. Thus, normality is defined in a very limited manner.

A human is considered “obese” if the body mass index (BMI) (weight divided by height squared) is at least 30 kg/m². A human who does not satisfy this standard may be characterized as “non-obese”, the term “normal” being reserved for use in reference to glucose and insulin levels as previously described.

A human is considered overweight if the BMI is at least 25 kg/m². Thus, we define overweight to include obese individuals, consistent with the recommendations of the National Institute of Diabetes and Digestive and Kidney Diseases (NIDDK). A human who does not satisfy this standard may be characterized as “non-overweight.”

According to the Report of the Expert Committe on the Diagnosis and Classification of Diabetes Mellitus, Diabetes Care 20: 1183-97 (1997), the following are risk factors for diabetes type II:
older (e.g., at least 45; see below)
excessive weight (see below)
first-degree relative with diabetes mellitus
member of high risk ethnic group (black, Hispanic, Native American, Asian)
history of gestational diabetes mellitus or delivering a baby weighing more than 9 pounds (4.032 kg)
hypertensive (>140/90 mm Hg)
HDL cholesterol level >35 mg/dL (0.90 mmol/L)
triglyceride level >=250 mg/dL (2.83 mmol/L)

Hence, in a preferred embodiment, the diagnostic and protective methods of the present invention are applied to human subjects exhibiting one or more of the aforementioned risk factors. Likewise, in a preferred embodiment, they are applied to human subjects who, while not diabetic, exhibit impaired glucose homeostasis (110 to <126 mg/dL).

The risk of diabetes increases with age. Hence, in successive preferred embodiments, the age of the subjects is at least 45, at least 50, at least 55, at least 60, at least 65, at least 70, and at least 75.

With regard to excessive weight, NIDDK says that “The relative risk of diabetes increases by approximately 25 percent for each additional unit of BMI over 22.” Hence, in successive preferred embodiments, the BMIs of the human subjects is at least 23, at least 24, at least 25 (i.e., overweight by our criterion), at least 26, at least 27, at least 28, at least 29, at least 30 (i.e., obese), at least 31, at least 32, at least 33, at least 34, at least 35, at least 36, at least 37, at least 38, at least 39, at least 40, or over 40.

Genes/Proteins of Interest

Favorable genes/proteins are those corresponding to genes less strongly expressed in hyperinsulinemic liver than in normal liver, or in type II diabetic liver as compared to hyperinsulinemic liver. Unfavorable genes/proteins are those corresponding to genes more strongly expressed in hyperinsulinemic liver than in normal liver, or in type II diabetic liver as compared to hyperinsulinemic liver.

Mixed genes/proteins are those exhibiting a combination of favorable and unfavorable behavior. A mixed gene/protein can be used as would a favorable gene/protein if its favorable behavior outweighs the unfavorable. It can be used as would an unfavorable gene/protein if its unfavorable behavior outweighs the favorable. Preferably, they are used in conjunction with other agents that affect their balance of favorable and unfavorable behavior. Use of mixed genes/proteins is, in general, less desirable than use of purely favorable or purely unfavorable genes/proteins.

For each of the differentially expressed genes, corresponding mouse and human proteins have been identified, as set forth in the Master Tables.

Direct and Indirect Utility of Identified Nucleic Acid

Sequences and Related Molecules

The mouse or human genes (or fragments thereof) may be used directly. For diagnostic or screening purposes, they (or specific binding fragments thereof) may be labeled and used as hybridization probes. For therapeutic purposes, they (or specific binding fragments thereof) may be used as antisense reagents to inhibit the expression of the corresponding gene, or of a sufficiently homologous gene of another species.

Since each of the probes is representative of a full-length mouse gene, that is, it encodes an entire, functional protein, then it may be used in the expression of that protein. Likewise, if the corresponding human gene is known in full-length, it may be used to express the human protein. Such expression may be in cell culture, with the protein subsequently isolated and administered exogenously to subjects who would benefit therefrom, or in vivo, i.e., administration by gene therapy. Naturally, any DNA encoding the same protein, or a fragment or a mutant protein which retains the desired activity, may be used for the same purpose. The encoded protein of course has utility therapeutically and, in labeled or immobilized form, diagnostically.

The genes may also be used indirectly, that is, to identify other useful DNAs, proteins, or other molecules.

There thus are several ways that a human protein homologue of interest can be identified by database searching, including:
1) a DNA->DNA (BlastN) search for database DNAs closely related to the mouse gene identifies a known human gene, and the sequence of the human protein is deduced by the Genetic Code;
2) a DNA->Protein (BlastX) search for database proteins closely related to the translated DNA of the mouse gene identifies a known human protein; and
3) the sequence of the mouse protein is known or is deduced by the Genetic Code, and a Protein->Protein (BlastP) search for closely related database proteins identifies a known human protein.

Once a known human gene is identified, it may be used in further BlastN or BlastX searches to identify other human genes or proteins. Once a known human protein is identified, it may be used in further BlastP searches to identify other human proteins.

Searches may also take cognizance, intermediately, of known genes and proteins other than mouse or human ones, e.g., use the mouse sequence to identify a known rat sequence and then the rat sequence to identify a human one.

Thus, if we have identified a mouse gene, and it encodes a mouse protein which appears similar to a human protein, then that human protein may be used (especially in humans) for purposes analogous to the proposed use of the mouse protein in mice. Moreover, a specific binding fragment of an appropriate strand of the corresponding human gene or cDNA could be labeled and used as a hybridization probe (especially against samples of human mRNA or cDNA).

In determining whether the disclosed genes have significant similarities to known DNAs (and their translated AA sequences to known proteins), one would generally use the disclosed gene as a query sequence in a search of a sequence database. The results of several such searches are set forth in the Examples. Such results are dependent, to some degree, on the search parameters. Preferred parameters are set forth in Example 1. The results are also dependent on the content of the database. While the raw similarity score of a particular target (database) sequence will not vary with content (as long as it remains in the database), its informational value (in bits), expected value, and relative ranking can change. Generally speaking, the changes are small.

It will be appreciated that the nucleic acid and protein databases keep growing. Hence a later search may identify high scoring target sequences which were not uncovered by an earlier search because the target sequences were not previously part of a database.

Hence, in a preferred embodiment, the cognate DNAs and proteins include not only those set forth in the examples, but those which would have been highly ranked (top ten, more preferably top three, even more preferably top two, most preferably the top one) in a search run with the same parameters on the date of filing of this application.

If the known human DNA is appears to be a partial DNA, it may be used as a hybridization probe to isolate the full-length DNA. If the partial DNA encodes a biologically functional fragment of the cognate protein, it may be used in a manner similar to the full length DNA, i.e., to produce the functional fragment.

If we have indicated that an antagonist of a protein or other molecule is useful, then such an antagonist may be obtained by preparing a combinatorial library, as described below, of potential antagonists, and screening the library members for binding to the protein or other molecule in question. The binding members may then be further screened for the ability to antagonize the biological activity of the target. The antagonists may be used therapeutically, or, in suitably labeled or immobilized form, diagnostically.

If the identified DNA is related to a known protein, then substances known to interact with that protein (e.g., agonists, antagonists, substrates, receptors, second messengers, regulators, and so forth), and binding molecules which bind them, are also of utility. Such binding molecules can likewise be identified by screening a combinatorial library.

Isolation of Full Length cDNAs Using Partial cDNAs as Probes

If it is determined that a DNA of the present invention is a partial DNA, and the cognate full length DNA is not listed in a sequence database, the available DNA may be used as a hybridization probe to isolate the full-length cDNA from a suitable cDNA library.

Stringent hybridization conditions are appropriate, that is, conditions in which the hybridization temperature is 5-10 deg. C. below the Tm of the cDNA as a perfect duplex.

Identification and Isolation of Homologous Genes/cDNAs Using a cDNA Probe

It may be that the sequence databases available do not include the sequence of any homologous gene, or at least of the homologous gene for a species of interest. However, given the cDNAs set forth above, one may readily obtain the homologous gene.

The possession of one DNA (the “starting DNA”) greatly facilitates the isolation of homologous genes/cDNAs. If only a partial DNA is known, this partial DNA may first be used as a probe to isolate the corresponding full length DNA for the same species, and that the latter may be used as the starting DNA in the search for homologous genes.

The starting DNA, or a fragment thereof, is used as a hybridization probe to screen a cDNA or genomic DNA library for clones containing inserts which encode either the entire homologous protein, or a recognizable fragment thereof. The minimum length of the hybridization probe is dictated by the need for specificity. If the size of the library in bases is L, and the GC content is 50%, then the probe should have a length of at least 1, where L=41. This will yield, on average, a single perfect match in random DNA of L bases. The human cDNA library is about 10⁸ bases and the human genomic DNA library is about 10¹⁰ bases.

The library is preferably derived from an organism which is known, on biochemical evidence, to produce a homologous protein, and more preferably from the genomic DNA or mRNA of cells of that organism which are likely to be relatively high producers of that protein. A cDNA library (which is derived from an mRNA library) is especially preferred.

If the organism in question is known to have substantially different codon preferences from that of the organism whose relevant cDNA or genomic DNA is known, a synthetic hybridization probe may be used which encodes the same amino acid sequence but whose codon utilization is more similar to that of the DNA of the target organism. Alternatively, the synthetic probe may employ inosine as a substitute for those bases which are most likely to be divergent, or the probe may be a mixed probe which mixes the codons for the source DNA with the preferred codons (encoding the same amino acid) for the target organism.

By routine methods, the Tm of a perfect duplex of starting DNA is determined. One may then select a hybridization temperature which is sufficiently lower than the perfect duplex Tm to allow hybridization of the starting DNA (or other probe) to a target DNA which is divergent from the starting DNA. A 1% sequence divergence typically lowers the Tm of a duplex by 1-2° C., and the DNAs encoding homologous proteins of different species typically have sequence identities of around 50-80%. Preferably, the library is screened under conditions where the temperature is at least 20° C., more preferably at least 50° C., below the perfect duplex Tm. Since salt reduces the Tm, one ordinarily would carry out the search for DNAs encoding highly homologous proteins under relatively low salt hybridization conditions, e.g., <1 M NaCl. The higher the salt concentration, and/or the lower the temperature, the greater the sequence divergence which is tolerated.

For the use of probes to identify homologous genes in other species, see, e.g., Schwinn, et al., J. Biol. Chem., 265:8183-89 (1990) (hamster 67-bp cDNA probe vs. human leukocyte genomic library; human 0.32 kb DNA probe vs. bovine brain cDNA library, both with hybridization at 42° C. in 6×SSC); Jenkins et al., J. Biol. Chem., 265:19624-31 (1990) (Chicken 770-bp cDNA probe vs. human genomic libraries; hybridization at 40° C. in 50% formamide and 5×SSC); Murata et al., J. Exp. Med., 175:341-51 (1992) (1.2-kb mouse cDNA probe v. human eosinophl cDNA library; hybridization at 65° C. in 6×SSC); Guyer et al., J. Biol. Chem., 265:17307-17 (1990) (2.95-kb human genomic DNA probe vs. porcine genomic DNA library; hybridization at 42° C. in 5×SSC). The conditions set forth in these articles may each be considered suitable for the purpose of isolating homologous genes.

Homologous Proteins and DNAs

A human protein can be said to be identifiable as homologous to a mouse gene (and hence to “correspond” to such gene) if
(1) its sequence can be aligned to the mouse gene, using BlastX with the default parameters set forth below, and the expected value (E) of the alignment (the probability that such an alignment would have occurred by chance alone) is less than e-10,
(2) its sequence can be aligned to a human gene, using BlastX with the default parameters set forth below, and the cDNA of said human gene can be aligned to the mouse gene, using BlastN with the default parameters set forth below, and the E value for both alignments is less than e-10,
(3) its sequence can be aligned to a mouse protein, using BlastP with the default parameters set forth below, and that mouse protein can be aligned to the mouse gene, using BlastX with the default parameters set forth below, and in both alignments the E value of the alignment is less than e-10.

Naturally, if the human protein is encoded by the human gene of (2), or the mouse protein is encoded by the mouse gene of (3), the BlastX alignment will be satisfied.

Desirably, two or all three of these conditions (1)-(3) are satisfied.

Preferably, for any of the alignments noted above, and more preferably for all of them, the E value is less than e-15, more preferably less than e-20, still more preferably less than e-40, even more preferably less than e-60, considerably more preferably less than e-80, and most preferably less than e-100. More preferably, for those conditions in which the mouse cDNA clone is indirectly connected to the human protein by virtue of two or more successive alignments, the E value is so limited for all of said alignments in the connecting chain.

BlastN and BlastX report very low expected values as “0.0”. This does not truly mean that the expected value is exactly zero (since any alignment could occur by chance), but merely that it is so infinitesimal that it is not reported. The documentation does not state the cutoff value, alignments with explicit E values as low as e-178 (624 bits) have been reported as such, while a score of 636 bits was reported as “0.0”.

Functionally homologous human proteins are also of interest. A human protein may be said to be functionally homologous to the mouse gene if (1)it can be aligned to the mouse gene, using BlastX with the default parameters set forth below, and the E value of the alignment is less than e-50, and (2) the human protein has at least one biological activity in common with the mouse protein.

The human proteins of interest also include those that are substantially and/or conservatively identical (as defined below) to the homologous and/or functionally homologous human proteins defined above.

Relevance of Favorable and Unfavorable Genes

If a gene is down-regulated in more favored mammals, or up-regulated in less favored mammals, (i.e., an “unfavorable gene”) then several utilities are apparent.

First, the complementary strand of the gene, or a portion thereof, may be used in labeled form as a hybridization probe to detect messenger RNA and thereby monitor the level of expression of the gene in a subject. Elevated levels are indicative of progression, or propensity to progression, to a less favored state, and clinicians may take appropriate preventative, curative or ameliorative action.

Secondly, the messenger RNA product (or equivalent cDNA), the protein product, or a binding molecule specific for that product (e.g., an antibody which binds the product), or a downstream product which mediates the activity (e.g., a signaling intermediate) or a binding molecule (e.g., an antibody) therefor, may be used, preferably in labeled or immobilized form, as an assay reagent in an assay for said nucleic acid product, protein product, or downstream product (e.g., a signaling intermediate). Again, elevated levels are indicative of a present or future problem.

Thirdly, an agent which down-regulates expression of the gene may be used to reduce levels of the corresponding protein and thereby inhibit further damage. This agent could inhibit transcription of the gene in the subject, or translation of the corresponding messenger RNA. Possible inhibitors of transcription and translation include antisense molecules and repressor molecules. The agent could also inhibit a post-translational modification (e.g., glycosylation, phosphorylation, cleavage, GPI attachment) required for activity, or post-translationally modify the protein so as to inactivate it. Or it could be an agent which down-or up-regulated a positive or negative regulatory gene, respectively.

Fourthly, an agent which is an antagonist of the messenger RNA product or protein product of the gene, or of a downstream product through which its activity is manifested (e.g., a signaling intermediate), may be used to inhibit its activity.

This antagonist could be an antibody, a peptide, a peptoid, a nucleic acid, a peptide nucleic acid (PNA) oligomer, a small organic molecule of a kind for which a combinatorial library exists (e.g., a benzodiazepine), etc. An antagonist is simply a binding molecule which, by binding, reduces or abolishes the undesired activity of its target. The antagonist, if not an oligomeric molecule, is preferably less than 500 daltons.

Fifthly, an agent which degrades, or abets the degradation of, that messenger RNA, its protein product or a downstream product which mediates its activity (e.g., a signaling intermediate), may be used to curb the effective period of activity of the protein.

If a gene is up-regulated in more favored mammals, or down-regulated in less favored animals then the utilities are converse to those stated above.

First, the complementary strand of the gene, or a portion thereof, may be used in labeled form as a hybridization probe to detect messenger RNA and thereby monitor the level of expression of the gene in a subject. Depressed levels are indicative of damage, or possibly of a propensity to damage, and clinicians may take appropriate preventative, curative or ameliorative action.

Secondly, the messenger RNA product, the equivalent cDNA, protein product, or a binding molecule specific for those products, or a downstream product, or a signaling intermediate, or a binding molecule therefor, may be used, preferably in labeled or immobilized form, as an assay reagent in an assay for said protein product or downstream product. Again, depressed levels are indicative of a present or future problem.

Thirdly, an agent which up-regulates expression of the gene may be used to increase levels of the corresponding protein and thereby inhibit further progression to a less favored state. By way of example, it could be a vector which carries a copy of the gene, but which expresses the gene at higher levels than does the endogenous expression system. Or it could be an agent which up-or down-regulates a positive or negative regulatory gene.

Fourthly, an agent which is an agonist of the protein product of the gene, or of a downstream product through which its activity (of inhibition of progression to a less favored state) is manifested, or of a signaling intermediate may be used to foster its activity.

Fifthly, an agent which inhibits the degradation of that protein product or of a downstream product or of a signaling intermediate may be used to increase the effective period of activity of the protein.

Mutant Proteins

The present invention also contemplates mutant proteins (peptides) which are substantially identical (as defined below) to the parental protein (peptide). In general, the fewer the mutations, the more likely the mutant protein is to retain the activity of the parental protein. The effect of mutations is usually (but not always) additive. Certain individual mutations are more likely to be tolerated than others.

A protein is more likely to tolerate a mutation which
(a) is a substitution rather than an insertion or deletion;
(b) is an insertion or deletion at the terminus, rather than internally, or, if internal, is at a domain boundary, or a loop or turn, rather than in an alpha helix or beta strand;
(c) affects a surface residue rather than an interior residue;
(d) affects a part of the molecule distal to the binding site;
(e) is a substitution of one amino acid for another of similar size, charge, and/or hydrophobicity, and does not destroy a disulfide bond or other crosslink; and
(f) is at a site which is subject to substantial variation among a family of homologous proteins to which the protein of interest belongs.
   These considerations can be used to design functional mutants.
   Surface vs. Interior Residues

Charged residues almost always lie on the surface of the protein. For uncharged residues, there is less certainty, but in general, hydrophilic residues are partitioned to the surface and hydrophobic residues to the interior. Of course, for a membrane protein, the membrane-spanning segments are likely to be rich in hydrophobic residues.

Surface residues may be identified experimentally by various labeling techniques, or by 3-D structure mapping techniques like X-ray diffraction and NMR. A 3-D model of a homologous protein can be helpful.

Binding Site Residues

Residues forming the binding site may be identified by (1) comparing the effects of labeling the surface residues before and after completing the protein to its target, (2) labeling the binding site directly with affinity ligands, (3) fragmenting the protein and testing the fragments for binding activity, and (4) systematic mutagenesis (e.g., alanine-scanning mutagenesis) to determine which mutants destroy binding. If the binding site of a homologous protein is known, the binding site may be postulated by analogy.

Protein libraries may be constructed and screened that a large family (e.g., 10⁸) of related mutants may be evaluated simultaneously.

Hence, the mutations are preferably conservative modifications as defined below.

“Substantially Identical”

A mutant protein (peptide) is substantially identical to a reference protein (peptide) if (a) it has at least 10% of a specific binding activity or a non-nutritional biological activity of the reference protein, and (b) is at least 50% identical in amino acid sequence to the reference protein (peptide). It is “substantially structurally identical” if condition (b) applies, regardless of (a).

Percentage amino acid identity is determined by aligning the mutant and reference sequences according to a rigorous dynamic programming algorithm which globally aligns their sequences to maximize their similarity, the similarity being scored as the sum of scores for each aligned pair according to an unbiased PAM250 matrix, and a penalty for each internal gap of −12 for the first null of the gap and 4 for each additional null of the same gap. The percentage identity is the number of matches expressed as a percentage of the adjusted (i.e., counting inserted nulls) length of the reference sequence.

A mutant DNA sequence is substantially identical to a reference DNA sequence if they are structural sequences, and encoding mutant and reference proteins which are substantially identical as described above.

If instead they are regulatory sequences, they are substantially identical if the mutant sequence has at least 10% of the regulatory activity of the reference sequence, and is at least 50% identical in nucleotide sequence to the reference sequence. Percentage identity is determined as for proteins except that matches are scored +5, mismatches −4, the gap open penalty is −12, and the gap extension penalty (per additional null) is −4.

Preferably, sequence which are substantially identical exceed the minimum identity of 50% e.g., are 51%, 66%, 75%, 80%, 85%, 90%, 95% or 99% identical in sequence.

DNA sequences may also be considered “substantially identical” if they hybridize to each other under stringent conditions, i.e., conditions at which the Tm of the heteroduplex of the one strand of the mutant DNA and the more complementary strand of the reference DNA is not in excess of 10° C. less than the Tm of the reference DNA homoduplex. Typically this will correspond to a percentage identity of 85-90%.

“Conservative Modifications”

“Conservative modifications” are defined as
(a) conservative substitutions of amino acids as hereafter defined; or
(b) single or multiple insertions (extension) or deletions (truncation) of amino acids at the termini.

Conservative modifications are preferred to other modifications. Conservative substitutions are preferred to other conservative modifications. “Semi-Conservative Modifications” are modifications which are not conservative, but which are (a) semi-conservative substitutions as hereafter defined; or (b) single or multiple insertions or deletions internally, but at interdomain boundaries, in loops or in other segments of relatively high mobility. Semi-conservative modifications are preferred to nonconservative modifications. Semi-conservative substitutions are preferred to other semi-conservative modifications.

Non-conservative substitutions are preferred to other non-conservative modifications.

The term “conservative” is used here in an a priori sense, i.e., modifications which would be expected to preserve 3D structure and activity, based on analysis of the naturally occurring families of homologous proteins and of past experience with the effects of deliberate mutagenesis, rather than post facto, a modification already known to conserve activity. Of course, a modification which is conservative a priori may, and usually is, also conservative post facto.

Preferably, except at the termini, no more than about five amino acids are inserted or deleted at a particular locus, and the modifications are outside regions known to contain binding sites important to activity.

Preferably, insertions or deletions are limited to the termini.

A conservative substitution is a substitution of one amino acid for another of the same exchange group, the exchange groups being defined as follows
I Gly, Pro, Ser, Ala (Cys) (and any nonbiogenic, neutral amino acid with a hydrophobicity not exceeding that of the aforementioned a.a.'s)
II Arg, Lys, His (and any nonbiogenic, positively-charged amino acids)
III Asp, Glu, Asn, Gln (and any nonbiogenic negatively-charged amino acids)
IV Leu, Ile, Met, Val (Cys) (and any nonbiogenic, aliphatic, neutral amino acid with a hydrophobicity too high for I above)
V Phe, Trp, Tyr (and any nonbiogenic, aromatic neutral amino acid with a hydrophobicity too high for I above).

Note that Cys belongs to both I and IV.

Residues Pro, Gly and Cys have special conformational roles. Cys participates in formation of disulfide bonds. Gly imparts flexibility to the chain. Pro imparts rigidity to the chain and disrupts a helices. These residues may be essential in certain regions of the polypeptide, but substitutable elsewhere.

One, two or three conservative substitutions are more likely to be tolerated than a larger number.

“Semi-conservative substitutions” are defined herein as being substitutions within supergroup I/II/III or within supergroup IV/V, but not within a single one of groups I-V. They also include replacement of any other amino acid with alanine. If a substitution is not conservative, it preferably is semi-conservative.

“Non-conservative substitutions” are substitutions which are not “conservative” or “semi-conservative”.

“Highly conservative substitutions” are a subset of conservative substitutions, and are exchanges of amino acids within the groups Phe/Tyr/Trp, Met/Leu/Ile/Val, His/Arg/Lys, Asp/Glu and Ser/Thr/Ala. They are more likely to be tolerated than other conservative substitutions. Again, the smaller the number of substitutions, the more likely they are to be tolerated.

“Conservatively Identical”

A protein (peptide) is conservatively identical to a reference protein (peptide) it differs from the latter, if at all, solely by conservative modifications, the protein (peptide remaining at least seven amino acids long if the reference protein (peptide) was at least seven amino acids long.

A protein is at least semi-conservatively identical to a reference protein (peptide) if it differs from the latter, if at all, solely by semi-conservative or conservative modifications.

A protein (peptide) is nearly conservatively identical to a reference protein (peptide) if it differs from the latter, if at all, solely by one or more conservative modifications and/or a single nonconservative substitution.

It is highly conservatively identical if it differs, if at all, solely by highly conservative substitutions. Highly conservatively identical proteins are preferred to those merely conservatively identical. An absolutely identical protein is even more preferred.

The core sequence of a reference protein (peptide) is the largest single fragment which retains at least 10% of a particular specific binding activity, if one is specified, or otherwise of at least one specific binding activity of the referent. If the referent has more than one specific binding activity, it may have more than one core sequence, and these may overlap or not.

If it is taught that a peptide of the present invention may have a particular similarity relationship (e.g., markedly identical) to a reference protein (peptide), preferred peptides are those which comprise a sequence having that relationship to a core sequence of the reference protein (peptide), but with internal insertions or deletions in either sequence excluded. Even more preferred peptides are those whose entire sequence has that relationship, with the same exclusion, to a core sequence of that reference protein (peptide).

Library

The term “library” generally refers to a collection of chemical or biological entities which are related in origin, structure, and/or function, and which can be screened simultaneously for a property of interest.

Libraries may be classified by how they are constructed (natural vs. artificial diversity; combinatorial vs. noncombinatorial), how they are screened (hybridization, expression, display), or by the nature of the screened library members (peptides, nucleic acids, etc.).

In a “natural diversity” library, essentially all of the diversity arose without human intervention. This would be true, for example, of messenger RNA extracted from a non-engineered cell.

In a “synthetic diversity” library, essentially all of the diversity arose deliberately as a result of human intervention. This would be true for example of a combinatorial library; note that a small level of natural diversity could still arise as a result of spontaneous mutation. It would also be true of a noncombinatorial library of compounds collected from diverse sources, even if they were all natural products.

In a “non-natural diversity” library, at least some of the diversity arose deliberately through human intervention.

In a “controlled origin” library, the source of the diversity is limited in some way. A limitation might be to cells of a particular individual, to a particular species, or to a particular genus, or, more complexly, to individuals of a particular species who are of a particular age, sex, physical condition, geographical location, occupation and/or familial relationship. Alternatively or additionally, it might be to cells of a particular tissue or organ. Or it could be cells exposed to particular pharmacological, environmental, or pathogenic conditions. Or the library could be of chemicals, or a particular class of chemicals, produced by such cells.

In a “controlled structure” library, the library members are deliberately limited by the production conditions to particular chemical structures. For example, if they are oligomers, they may be limited in length and monomer composition, e.g. hexapeptides composed of the twenty genetically encoded amino acids.

Hybridization Library

In a hybridization library, the library members are nucleic acids, and are screened using a nucleic acid hybridization probe. Bound nucleic acids may then be amplified, cloned, and/or sequenced.

Expression Library

In an expression library, the screened library members are gene expression products, but one may also speak of an underlying library of genes encoding those products. The library is made by subcloning DNA encoding the library members (or portions thereof) into expression vectors (or into cloning vectors which subsequently are used to construct expression vectors), each vector comprising an expressible gene encoding a particular library member, introducing the expression vectors into suitable cells, and expressing the genes so the expression products are produced.

In one embodiment, the expression products are secreted, so the library can be screened using an affinity reagent, such as an antibody or receptor. The bound expression products may be sequenced directly, or their sequences inferred by, e.g., sequencing at least the variable portion of the encoding DNA.

In a second embodiment, the cells are lysed, thereby exposing the expression products, and the latter are screened with the affinity reagent.

In a third embodiment, the cells express the library members in such a manner that they are displayed on the surface of the cells, or on the surface of viral particles produced by the cells. (See display libraries, below).

In a fourth embodiment, the screening is not for the ability of the expression product to bind to an affinity reagent, but rather for its ability to alter the phenotype of the host cell in a particular detectable manner. Here, the screened library members are transformed cells, but there is a first underlying library of expression products which mediate the behavior of the cells, and a second underlying library of genes which encode those products.

Display Library

In a display library, the library members are each conjugated to, and displayed upon, a support of some kind. The support may be living (a cell or virus), or nonliving (e.g., a bead or plate).

If the support is a cell or virus, display will normally be effectuated by expressing a fusion protein which comprises the library member, a carrier moiety allowing integration of the fusion protein into the surface of the cell or virus, and optionally a lining moiety. In a variation on this theme, the cell coexpresses a first fusion comprising the library member and a linking moiety L1, and a second fusion comprising a linking moiety L2 and the carrier moiety. L1 and L2 interact to associate the first fusion with the second fusion and hence, indirectly, the library member with the surface of the cell or virus.

Soluble Library

In a soluble library, the library members are free in solution. A soluble library may be produced directly, or one may first make a display library and then release the library members from their supports.

Encapsulated Library

In an encapsulated library, the library members are inside cells or liposomes. Generally speaking, encapsulated libraries are used to store the library members for future use; the members are extracted in some way for screening purposes. However, if they differentially affect the phenotype of the cells, they may be screened indirectly by screening the cells.

cDNA Library

A cDNA library is usually prepared by extracting RNA from cells of particular origin, fractionating the RNA to isolate the messenger RNA (mRNA has a poly(A) tail, so this is usually done by oligo-dT affinity chromatography), synthesizing complementary DNA (cDNA) using reverse transcriptase, DNA polymerase, and other enzymes, subcloning the cDNA into vectors, and introducing the vectors into cells. Often, only mRNAs or cDNAs of particular sizes will be used, to make it more likely that the cDNA encodes a functional polypeptide.

A cDNA library explores the natural diversity of the transcribed DNAs of cells from a particular source. It is not a combinatorial library.

A cDNA library may be used to make a hybridization library, or it may be used as an (or to make) expression library.

Genomic DNA Library

A genomic DNA library is made by extracting DNA from a particular source, fragmenting the DNA, isolating fragments of a particular size range, subcloning the DNA fragments into vectors, and introducing the vectors into cells.

Like a cDNA library, a genomic DNA library is a natural diversity library, and not a combinatorial library. A genomic DNA library may be used the same way as a cDNA library.

Synthetic DNA library

A synthetic DNA library may be screened directly (as a hybridization library), or used in the creation of an expression or display library of peptides/proteins.

Combinatorial Libraries

The term “combinatorial library” refers to a library in which the individual members are either systematic or random combinations of a limited set of basic elements, the properties of each member being dependent on the choice and location of the elements incorporated into it. Typically, the members of the library are at least capable of being screened simultaneously. Randomization may be complete or partial; some positions may be randomized and others predetermined, and at random positions, the choices may be limited in a predetermined manner. The members of a combinatorial library may be oligomers or polymers of some kind, in which the variation occurs through the choice of monomeric building block at one or more positions of the oligomer or polymer, and possibly in terms of the connecting linkage, or the length of the oligomer or polymer, too. Or the members may be nonoligomeric molecules with a standard core structure, like the 1,4-benzodiazepine structure, with the variation being introduced by the choice of substituents at particular variable sites on the core structure. Or the members may be nonoligomeric molecules assembled like a jigsaw puzzle, but wherein each piece has both one or more variable moieties (contributing to library diversity) and one or more constant moieties (providing the functionalities for coupling the piece in question to other pieces).

Thus, in a typical combinatorial library, chemical building blocks are at least partially randomly combined into a large number (as high as 10¹⁵) of different compounds, which are then simultaneously screened for binding (or other) activity against one or more targets.

In a “simple combinatorial library”, all of the members belong to the same class of compounds (e.g., peptides) and can be synthesized simultaneously. A “composite combinatorial library” is a mixture of two or more simple libraries, e.g., DNAs and peptides, or peptides, peptoids, and PNAS, or benzodiazepines and carbamates. The number of component simple libraries in a composite library will, of course, normally be smaller than the average number of members in each simple library, as otherwise the advantage of a library over individual synthesis is small.

Libraries of thousands, even millions, of random oligopeptides have been prepared by chemical synthesis (Houghten et al., Nature, 354:84-6(1991)), or gene expression (Marks et al., J Mol Biol, 222:581-97(1991)), displayed on chromatographic supports (Lam et al., Nature, 354:82-4(1991)), inside bacterial cells (Colas et al., Nature, 380:548-550(1996)), on bacterial pili (Lu, Bio/Technology, 13:366-372(1990)), or phage (Smith, Science, 228:1315-7(1985)), and screened for binding to a variety of targets including antibodies (Valadon et al., J Mol Biol, 261:11-22(1996)), cellular proteins (Schmitz et al., J Mol Biol, 260:664-677(1996)), viral proteins (Hong and Boulanger, Embo J, 14:4714-4727(1995)), bacterial proteins (Jacobsson and Frykberg, Biotechniques, 18:878-885(1995)), nucleic acids (Cheng et al., Gene, 171:1-8(1996)), and plastic (Siani et al., J Chem Inf Comput Sci, 34:588-593(1994)).

Libraries of proteins (Ladner, U.S. Pat. No. 4,664,989), peptoids (Simon et al., Proc Natl Acad Sci USA, 89:9367-71(1992)), nucleic acids (Ellington and Szostak, Nature, 246:818(1990)), carbohydrates, and small organic molecules (Eichler et al., Med Res Rev, 15:481-96(1995)) have also been prepared or suggested for drug screening purposes.

The first combinatorial libraries were composed of peptides or proteins, in which all or selected amino acid positions were randomized. Peptides and proteins can exhibit high and specific binding activity, and can act as catalysts. In consequence, they are of great importance in biological systems.

Nucleic acids have also been used in combinatorial libraries. Their great advantage is the ease with which a nucleic acid with appropriate binding activity can be amplified. As a result, combinatorial libraries composed of nucleic acids can be of low redundancy and hence, of high diversity.

There has also been much interest in combinatorial libraries based on small molecules, which are more suited to pharmaceutical use, especially those which, like benzodiazepines, belong to a chemical class which has already yielded useful pharmacological agents. The techniques of combinatorial chemistry have been recognized as the most efficient means for finding small molecules that act on these targets. At present, small molecule combinatorial chemistry involves the synthesis of either pooled or discrete molecules that present varying arrays of functionality on a common scaffold. These compounds are grouped in libraries that are then screened against the target of interest either for binding or for inhibition of biological activity.

The size of a library is the number of molecules in it. The simple diversity of a library is the number of unique structures in it. There is no formal minimum or maximum diversity. If the library has a very low diversity, the library has little advantage over just synthesizing and screening the members individually. If the library is of very high diversity, it may be inconvenient to handle, at least without automatizing the process. The simple diversity of a library is preferably at least 10, 10E2, 10E3, 10E4, 10E6, 10E7, 10E8 or 10E9, the higher the better under most circumstances. The simple diversity is usually not more than 10E15, and more usually not more than 10E10.

The average sampling level is the size divided by the simple diversity. The expected average sampling level must be high enough to provide a reasonable assurance that, if a given structure were expected, as a consequence of the library design, to be present, that the actual average sampling level will be high enough so that the structure, if satisfying the screening criteria, will yield a positive result when the library is screened. Thus, the preferred average sampling level is a function of the detection limit, which in turn is a function of the strength of the signal to be screened.

There are more complex measures of diversity than simple diversity. These attempt to take into account the degree of structural difference between the various unique sequences. These more complex measures are usually used in the context of small organic compound libraries, see below.

The library members may be presented as solutes in solution, or immobilized on some form of support. In the latter case, the support may be living (cell, virus) or nonliving (bead, plate, etc.). The supports may be separable (cells, virus particles, beads) so that binding and nonbinding members can be separated, or nonseparable (plate). In the latter case, the members will normally be placed on addressable positions on the support. The advantage of a soluble library is that there is no carrier moiety that could interfere with the binding of the members to the support. The advantage of an immobilized library is that it is easier to identify the structure of the members which were positive.

When screening a soluble library, or one with a separable support, the target is usually immobilized. When screening a library on a nonseparable support, the target will usually be labeled.

Oligonucleotide Libraries

An oligonucleotide library is a combinatorial library, at least some of whose members are single-stranded oligonucleotides having three or more nucleotides connected by phosphodiester or analogous bonds. The oligonucleotides may be linear, cyclic or branched, and may include non-nucleic acid moieties. The nucleotides are not limited to the nucleotides normally found in DNA or RNA. For examples of nucleotides modified to increase nuclease resistance and chemical stability of aptamers, see Chart 1 in Osborne and Ellington, Chem. Rev., 97: 349-70 (1997). For screening of RNA, see Ellington and Szostak, Nature, 346: 818-22 (1990).

There is no formal minimum or maximum size for these oligonucleotides. However, the number of conformations which an oligonucleotide can assume increases exponentially with its length in bases. Hence, a longer oligonucleotide is more likely to be able to fold to adapt itself to a protein surface. On the other hand, while very long molecules can be synthesized and screened, unless they provide a much superior affinity to that of shorter molecules, they are not likely to be found in the selected population, for the reasons explained by Osborne and Ellington (1997). Hence, the libraries of the present invention are preferably composed of oligonucleotides having a length of 3 to 100 bases, more preferably 15 to 35 bases. The oligonucleotides in a given library may be of the same or of different lengths.

Oligonucleotide libraries have the advantage that libraries of very high diversity (e.g., 10¹⁵) are feasible, and binding molecules are readily amplified in vitro by polymerase chain reaction (PCR). Moreover, nucleic acid molecules can have very high specificity and affinity to targets.

In a preferred embodiment, this invention prepares and screens oligonucleotide libraries by the SELEX method, as described in King and Famulok, Molec. Biol. Repts., 20: 97-107 (1994); L. Gold, C. Tuerk. Methods of producing nucleic acid ligands, U.S. Pat. No. 5,595,877; Oliphant et al. Gene 44:177 (1986).

The term “aptamer” is conferred on those oligonucleotides which bind the target protein. Such aptamers may be used to characterize the target protein, both directly (through identification of the aptamer and the points of contact between the aptamer and the protein) and indirectly (by use of the aptamer as a ligand to modify the chemical reactivity of the protein).

In a classic oligonuclotide, each nucleotide (monomeric unit) is composed of a phosphate group, a sugar moiety, and either a purine or a pyrimidine base. In DNA, the sugar is deoxyribose and in RNA it is ribose. The nucleotides are linked by 5′-3′ phosphodiester bonds.

The deoxyribose phosphate backbone of DNA can be modified to increase resistance to nuclease and to increase penetration of cell membranes. Derivatives such as mono-or dithiophosphates, methyl phosphonates, boranophosphates, formacetals, carbamates, siloxanes, and dimethylenethio- -sulfoxideo-and-sulfono-linked species are known in the art.

Peptide Library

A peptide is composed of a plurality of amino acid residues joined together by peptidyl (—NHCO—) bonds. A biogenic peptide is a peptide in which the residues are all genetically encoded amino acid residues; it is not necessary that the biogenic peptide actually be produced by gene expression.

Amino acids are the basic building blocks with which peptides and proteins are constructed. Amino acids possess both an amino group (—NH₂) and a carboxylic acid group (—COOH). Many amino acids, but not all, have the alpha amino acid structure NH₂—CHR—COOH, where R is hydrogen, or any of a variety of functional groups.

Twenty amino acids are genetically encoded: Alanine, Arginine, Asparagine, Aspartic Acid, Cysteine, Glutamic Acid, Glutamine, Glycine, Histidine, Isoleucine, Leucine, Lysine, Methionine, Phenylalanine, Proline, Serine, Threonine, Tryptophan, Tyrosine, and Valine. Of these, all save Glycine are optically isomeric, however, only the L-form is found in humans. Nevertheless, the D-forms of these amino acids do have biological significance; D-Phe, for example, is a known analgesic.

Many other amino acids are also known, including: 2-Aminoadipic acid; 3-Aminoadipic acid; beta-Aminopropionic acid; 2-Aminobutyric acid; 4-Aminobutyric acid (Piperidinic acid);6-Aminocaproic acid; 2-Aminoheptanoic acid; 2-Aminoisobutyric acid, 3-Aminoisobutyric acid; 2-Aminopimelic acid; 2,4-Diaminobutyric acid; Desmosine; 2,2′-Diaminopimelic acid; 2,3-Diaminopropionic acid; N-Ethylglycine; N-Ethylasparagine; Hydroxylysine; allo-Hydroxlysine; 3-Hydroxyproline; 4-Hydroxyproline; Isodesmosine; allo-Isoleucine; N-Methylglycine (Sarcosine); N-Methylisoleucine; N-Methylvaline; Norvaline; Norleucine; and Ornithine.

Peptides are constructed by condensation of amino acids and/or smaller peptides. The amino group of one amino acid (or peptide) reacts with the carboxylic acid group of a second amino acid (or peptide) to form a peptide (—NHCO—) bond, releasing one molecule of water. Therefore, when an amino acid is incorporated into a peptide, it should, technically speaking, be referred to as an amino acid residue. The core of that residue is the moiety which excludes the —NH and —CO linking functionalities which connect it to other residues. This moiety consists of one or more main chain atoms (see below) and the attached side chains.

The main chain moiety of each amino acid consists of the —NH and —CO linking functionalities and a core main chain moiety. Usually the latter is a single carbon atom. However, the core main chain moiety may include additional carbon atoms, and may also include nitrogen, oxygen or sulfur atoms, which together form a single chain. In a preferred embodiment, the core main chain atoms consist solely of carbon atoms.

The side chains are attached to the core main chain atoms. For alpha amino acids, in which the side chain is attached to the alpha carbon, the C-1, C-2 and N-2 of each residue form the repeating unit of the main chain, and the word “side chain” refers to the C-3 and higher numbered carbon atoms and their substituents. It also includes H atoms attached to the main chain atoms.

Amino acids may be classified according to the number of carbon atoms which appear in the main chain between the carbonyl carbon and amino nitrogen atoms which participate in the peptide bonds. Among the 150 or so amino acids which occur in nature, alpha, beta, gamma and delta amino acids are known. These have 1-4 intermediary carbons. Only alpha amino acids occur in proteins. Proline is a special case of an alpha amino acid; its side chain also binds to the peptide bond nitrogen.

For beta and higher order amino acids, there is a choice as to which main chain core carbon a side chain other than H is attached to. The preferred attachment site is the C-2 (alpha) carbon, i.e., the one adjacent to the carboxyl carbon of the —CO linking functionality. It is also possible for more than one main chain atom to carry a side chain other than H. However, in a preferred embodiment, only one main chain core atom carries a side chain other than H.

A main chain carbon atom may carry either one or two side chains; one is more common. A side chain may be attached to a main chain carbon atom by a single or a double bond; the former is more common.

A simple combinatorial peptide library is one whose members are peptides having three or more amino acids connected via peptide bonds.

The peptides may be linear, branched, or cyclic, and may covalently or noncovalently include nonpeptidyl moieties. The amino acids are not limited to the naturally occurring or to the genetically encoded amino acids.

A biased peptide library is one in which one or more (but not all) residues of the peptides are constant residues.

Cyclic Peptides

Many naturally occurring peptides are cyclic. Cyclization is a common mechanism for stabilization of peptide conformation thereby achieving improved association of the peptide with its ligand and hence improved biological activity. Cyclization is usually achieved by intra-chain cystine formation, by formation of peptide bond between side chains or between N- and C- terminals. Cyclization was usually achieved by peptides in solution, but several publications have appeared that describe cyclization of peptides on beads. A peptide library may be an oligopeptide library or a protein library.

Oligopeptides

Preferably, the oligopeptides are at least five, six, seven or eight amino acids in length. Preferably, they are composed of less than 50, more preferably less than 20 amino acids.

In the case of an oligopeptide library, all or just some of the residues may be variable. The oligopeptide may be unconstrained, or constrained to a particular conformation by, e.g., the participation of constant cysteine residues in the formation of a constraining disulfide bond.

Proteins

Proteins, like oligopeptides, are composed of a plurality of amino acids, but the term protein is usually reserved for longer peptides, which are able to fold into a stable conformation. A protein may be composed of two or more polypeptide chains, held together by covalent or noncovalent crosslinks. These may occur in a homooligomeric or a heterooligomeric state.

A peptide is considered a protein if it (1) is at least. 50 amino acids long, or (2) has at least two stabilizing covalent crosslinks (e.g., disulfide bonds). Thus, conotoxins are considered proteins.

Usually, the proteins of a protein library will be characterizable as having both constant residues (the same for all proteins in the library) and variable residues (which vary from member to member). This is simply because, for a given range of variation at each position, the sequence space (simple diversity) grows exponentially with the number of residue positions, so at some point it becomes inconvenient for all residues of a peptide to be variable positions. Since proteins are usually larger than oligopeptides, it is more common for protein libraries than oligopeptide libraries to feature variable positions.

In the case of a protein library, it is desirable to focus the mutations at those sites which are tolerant of mutation. These may be determined by alanine scanning mutagenesis or by comparison of the protein sequence to that of homologous proteins of similar activity. It is also more likely that mutation of surface residues will directly affect binding. Surface residues may be determined by inspecting a 3D structure of the protein, or by labeling the surface and then ascertaining which residues have received labels. They may also be inferred by identifying regions of high hydrophilicity within the protein.

Because proteins are often altered at some sites but not others, protein libraries can be considered a special case of the biased peptide library.

There are several reasons that one might screen a protein library instead of an oligopeptide library, including (1) a particular protein, mutated in the library, has the desired activity to some degree already, and (2) the oligopeptides are not expected to have a sufficiently high affinity or specificity since they do not have a stable conformation.

When the protein library is based on a parental protein which does not have the desired activity, the parental protein will usually be one which is of high stability (melting point >=50 deg. C.) and/or possessed of hypervariable regions.

The variable domains of an antibody possess hypervariable regions and hence, in some embodiments, the protein library comprises members which comprise a mutant of VH or VL chain, or a mutant of an antigen-specific binding fragment of such a chain. VH and VL chains are usually each about 110 amino acid residues, and are held in proximity by a disulfide bond between the adjoing CL and CH1 regions to form a variable domain. Together, the VH, VL, CL and CH1 form an Fab fragment.

In human heavy chains, the hypervariable regions are at 31-35, 49-65, 98-111 and 84-88, but only the first three are involved in antigen binding. There is variation among VH and VL chains at residues outside the hypervariable regions, but to a much lesser degree.

A sequence is considered a mutant of a VH or VL chain if it is at least 80% identical to a naturally occurring VH or VL chain at all residues outside the hypervariable region.

In a preferred embodiment, such antibody library members comprise both at least one VH chain and at least one VL chain, at least one of which is a mutant chain, and which chains may be derived from the same or different antibodies. The VH and VL chains may be covalently joined by a suitable linker moiety, as in a “single chain antibody”, or they may be noncovalently joined, as in a naturally occurring variable domain.

If the joining is noncovalent, and the library is displayed on cells or virus, then either the VH or the VL chain may be fused to the carrier surface/coat protein. The complementary chain may be co-expressed, or added exogenously to the library.

The members may further comprise some or all of an antibody constant heavy and/or constant light chain, or a mutant thereof.

Peptoid Library

A peptoid is an analogue of a peptide in which one or more of the peptide bonds (-NH-CO-) are replaced by pseudopeptide bonds, which may be the same or different. It is not necessary that all of the peptide bonds be replaced, i.e., a peptoid may include one or more conventional amino acid residues, e.g., proline.

A peptide bond has two small divalent linker elements, -NH- and -CO-. Thus, a preferred class of psuedopeptide bonds are those which consist of two small divalent linker elements. Each may be chosen independently from the group consisting of amine (—NH—), substituted amine (—NR—), carbonyl (—CO—), thiocarbonyl (—CS—),methylene (—CH2—), monosubstituted methylene (—CHR—), disubstituted methylene (—CR1R2—), ether (—O—) and thioether (—S—). The more preferred pseudopeptide bonds include:
N-modified —NRCO—
Carba Ψ—CH₂—CH₂—
Depsi Ψ—CO—O—
Hydroxyethylene Ψ—CHOH—CH₂—
Ketomethylene Ψ—CO—CH₂—
Methylene-Oxy —CH₂—O—
Reduced —CH₂—NH—
Thiomethylene —CH₂—S—
Thiopeptide —CS—NH—
Retro-Inverso —CO—NH—

A single peptoid molecule may include more than one kind of pseudopeptide bond.

For the purposes of introducing diversity into a peptoid library, one may vary (1) the side chains attached to the core main chain atoms of the monomers linked by the pseudopeptide bonds, and/or (2) the side chains (e.g., the —R of an —NRCO—) of the pseudopeptide bonds. Thus, in one embodiment, the monomeric units which are not amino acid residues are of the structure —NR1—CR2—CO—, where at least one of R1 and R2 are not hydrogen. If there is variability in the pseudopeptide bond, this is most conveniently done by using an —NRCO—or other pseudopeptide bond with an R group, and varying the R group. In this event, the R group will. usually be any of the side chains characterizing the amino acids of peptides, as previously discussed.

If the R group of the pseudopeptide bond is not variable, it will usually be small, e.g., not more than 10 atoms (e.g., hydroxyl, amino, carboxyl, methyl, ethyl, propyl).

If the conjugation chemistries are compatible, a simple combinatorial library may include both peptides and peptoids.

Peptide Nucleic Acid Library

A PNA oligomer is here defined as one comprising a plurality of units, at least one of which is a PNA monomer which comprises a side chain comprising a nucleobase. For nucleobases, see U.S. Pat. No. 6,077,835.

The classic PNA oligomer is composed of (2-aminoethyl) glycine units, with nucleobases attached by methylene carbonyl linkers. That is, it has the structure
H—(—HN-CH₂—CH₂—N(—CO—CH₂—B) —CH₂—CO—)ₙ —OH
where the outer parenthesized substructure is the PNA monomer.

In this structure, the nucleobase B is separated from the backbone N by three bonds, and the points of attachment of the side chains are separated by six bonds. The nucleobase may be any of the bases included in the nucleotides discussed in connection with oligonucleotide libraries. The bases of nucleotides A, G, T, C and U are preferred.

A PNA oligomer may further comprise one or more amino acid residues, especially glycine and proline.

One can readily envision related molecules in which (1) the —COCH2- linker is replaced by another linker, especially one composed of two small divalent linkers as defined previously, (2) a side chain is attached to one of the three main chain carbons not participating in the peptide bond (either instead or in addition to the side chain attached to the N of the classic PNA); and/or (3) the peptide bonds are replaced by pseudopeptide bonds as disclosed previously in the context of peptoids.

PNA oligomer libraries have been made; see e.g. Cook, U.S. Pat. No. 6,204,326.

Small Organic Compound Library

The small organic compound library (“compound library”, for short) is a combinatorial library whose members are suitable for use as drugs if, indeed, they have the ability to mediate a biological activity of the target protein.

Peptides have certain disadvantages as drugs. These include susceptibility to degradation by serum proteases, and difficulty in penetrating cell membranes. Preferably, all or most of the compounds of the compound library avoid, or at least do not suffer to the same degree, one or more of the pharmaceutical disadvantages of peptides.

In designing a compound library, it is helpful to bear in mind the methods of molecular modification typically used to obtain new drugs. Three basic kinds of modification may be identified: disjunction, in which a lead drug is simplified to identify its component pharmacophoric moieties; conjunction, in which two or more known pharmacophoric moieties, which may be the same or different, are associated, covalently or noncovalently, to form a new drug; and alteration, in which one moiety is replaced by another which may be similar or different, but which is not in effect a disjunction or conjunction. The use of the terms “disjunction”, “conjunction” and “alteration” is intended only to connote the structural relationship of the end product to the original leads, and not how the new drugs are actually synthesized, although it is possible that the two are the same.

The process of disjunction is illustrated by the evolution of neostigmine (1931) and edrophonium (1952) from physostigmine (1925). Subsequent conjunction is illustrated by demecarium (1956) and ambenonium (1956).

Alterations may modify the size, polarity, or electron distribution of an original moiety. Alterations include ring closing or opening, formation of lower or higher homologues, introduction or saturation of double bonds, introduction of optically active centers, introduction, removal or replacement of bulky groups, isosteric or bioisosteric substitution, changes in the position or orientation of a group, introduction of alkylating groups, and introduction, removal or replacement of groups with a view toward inhibiting or promoting inductive (electrostatic) or conjugative (resonance) effects.

Thus, the substituents may include electron acceptors and/or electron donors. Typical electron donors (+I) include —CH₃, —CH₂R, —CHR₂, —CR₃ and —COO−. Typical electron acceptors (—I) include —NH₃+, —NR₃+, —NO₂, —CN, —COOH, —COOR, —CHO, —COR, —COR, —F, —Cl, —Br, —OH, —OR, —SH, —SR, —CH═CH₂, —CR═CR₂, and —C═CH.

The substituents may also include those which increase or decrease electronic density in conjugated systems. The former (+R) groups include —CH₃, —CR₃, —F, —Cl, —Br, —I, —OH, —OR, —OCOR, —SH, —SR, —NH₂, —NR₂, and —NHCOR. The later (—R) groups include —NO₂, —CN, —CHC, —COR, —COOH, —COOR, —CONH₂, —SO₂R and —CF₃.

Synthetically speaking, the modifications may be achieved by a variety of unit processes, including nucleophilic and electrophilic substitution, reduction and oxidation, addition elimination, double bond cleavage, and cyclization.

For the purpose of constructing a library, a compound, or a family of compounds, having one or more pharmacological activities (which need not be related to the known or suspected activities of the target protein), may be disjoined into two or more known or potential pharmacophoric moieties. Analogues of each of these moieties may be identified, and mixtures of these analogues reacted so as to reassemble compounds which have some similarity to the original lead compound. It is not necessary that all members of the library possess moieties analogous to all of the moieties of the lead compound.

The design of a library may be illustrated by the example of the benzodiazepines. Several benzodiazepine drugs, including chlordiazepoxide, diazepam and oxazepam, have been used as anti-anxiety drugs. Derivatives of benzodiazepines have widespread biological activities; derivatives have been reported to act not only as anxiolytics, but also as anticonvulsants; cholecystokinin (CCK) receptor subtype A or B, kappa opioid receptor, platelet activating factor, and HIV transactivator Tat antagonists, and GPIIbIIa, reverse transcriptase and ras farnesyltransferase inhibitors.

The benzodiazepine structure has been disjoined into a 2-aminobenzophenone, an amino acid, and an alkylating agent. See Bunin, et al., Proc. Nat. Acad. Sci. USA, 91:4708 (1994). Since only a few 2-aminobenzophenone derivatives are commercially available, it was later disjoined into 2-aminoarylstannane, an acid chloride, an amino acid, and an alkylating agent. Bunin, et al., Meth. Enzymol., 267:448 (1996). The arylstannane may be considered the core structure upon which the other moieties are substituted, or all four may be considered equals which are conjoined to make each library member.

A basic library synthesis plan and member structure is shown in FIG. 1 of Fowlkes, et al., U.S. Ser. No. 08/740,671, incorporated by reference in its entirety. The acid chloride building block introduces variability at the R¹ site. The R² site is introduced by the amino acid, and the R³ site by the alkylating agent. The R⁴ site is inherent in the arylstannane. Bunin, et al. generated a 1, 4-benzodiazepine library of 11,200 different derivatives prepared from 20 acid chlorides, 35 amino acids, and 16 alkylating agents. (No diversity was introduced at R⁴; this group was used to couple the molecule to a solid phase.) According to the Available Chemicals Directory (HDL Information Systems, San Leandro Calif.), over 300 acid chlorides, 80 Fmoc-protected amino acids and 800 alkylating agents were available for purchase (and more, of course, could be synthesized). The particular moieties used were chosen to maximize structural dispersion, while limiting the numbers to those conveniently synthesized in the wells of a microtiter plate. In choosing between structurally similar compounds, preference was given to the least substituted compound.

The variable elements included both aliphatic and aromatic groups. Among the aliphatic groups, both acyclic and cyclic (mono- or poly-) structures, substituted or not, were tested. (While all of the acyclic groups were linear, it would have been feasible to introduce a branched aliphatic). The aromatic groups featured either single and multiple rings, fused or not, substituted or not, and with heteroatoms or not. The secondary substitutents included —NH₂, —OH, —OMe, —CN, —Cl, —F, and —COOH. While not used, spacer moieties, such as —O—, —S—, —OO—, —CS—, —NH—, and —NR—, could have been incorporated.

Bunin et al. suggest that instead of using a 1, 4-benzodiazepine as a core structure, one may instead use a 1, 4-benzodiazepine-2, 5-dione structure.

As noted by Bunin et al., it is advantageous, although not necessary, to use a linkage strategy which leaves no trace of the linking functionality, as this permits construction of a more diverse library.

Other combinatorial nonoligomeric compound libraries known or suggested in the art have been based on carbamates, mercaptoacylated pyrrolidines, phenolic agents, aminimides, N-acylamino ethers (made from amino alcohols, aromatic hydroxy acids, and carboxylic acids), N-alkylamino ethers (made from aromatic hydroxy acids, amino alcohols and aldehydes) 1, 4-piperazines, and 1, 4-piperazine-6-ones.

DeWitt, et al., Proc. Nat. Acad. Sci. (USA), 90:6909-13 (1993) describe the simultaneous but separate, synthesis of 40 discrete hydantoins and 40 discrete benzodiazepines. They carry out their synthesis on a solid support (inside a gas dispersion tube), in an array format, as opposed to other conventional simultaneous synthesis techniques (e.g., in a well, or on a pin). The hydantoins were synthesized by first simultaneously deprotecting and then treating each of five amino acid resins with each of eight isocyanates. The benzodiazepines were synthesized by treating each of five deprotected amino acid resins with each of eight 2-amino benzophenone imines.

Chen, et al., J. Am. Chem. Soc., 116:2661-62 (1994) described the preparation of a pilot (9 member) combinatorial library of formate esters. A polymer bead-bound aldehyde preparation was “split” into three aliquots, each reacted with one of three different ylide reagents. The reaction products were combined, and then divided into three new aliquots, each of which was reacted with a different Michael donor. Compound identity was found to be determinable on a single bead basis by gas chromatography/mass spectroscopy analysis.

Holmes, U.S. Pat. No. 5,549,974 (1996) sets forth methodologies for the combinatorial synthesis of libraries of thiazolidinones and metathiazanones. These libraries are made by combination of amines, carbonyl compounds, and thiols under cyclization conditions.

Ellman, U.S. Pat. No. 5,545,568 (1996) describes combinatorial synthesis of benzodiazepines, prostaglandins, beta-turn mimetics, and glycerol-based compounds. See also Ellman, U.S. Pat. No. 5,288,514.

Summerton, U.S. Pat. No. 5,506,337 (1996) discloses methods of preparing a combinatorial library formed predominantly of morpholino subunit structures.

Heterocylic combinatorial libraries are reviewed generally in Nefzi, et al.;, Chem. Rev., 97:449-472 (1997).

For pharmacological classes, see, e.g., Goth, Medical Pharmacology: Principles and Concepts (C. V. Mosby Co.: 8th ed. 1976); Korolkovas and Burckhalter, Essentials of Medicinal Chemistry (John Wiley & Sons, Inc.: 1976). For synthetic methods, see, e.g., Warren, Organic Synthesis: The Disconnection Approach (John Wiley & Sons, Ltd.: 1982); Fuson, Reactions of Organic Compounds (John Wiley & Sons: 1966); Payne and Payne, How to do an Organic Synthesis (Allyn and Bacon, Inc.: 1969); Greene, Protective Groups in Organic Synthesis (Wiley-Interscience). For selection of substituents, see e.g., Hansch and Leo, Substituent Constants for Correlation Analysis in Chemistry and Biology (John Wiley & Sons: 1979).

The library is preferably synthesized so that the individual members remain identifiable so that, if a member is shown to be active, it is not necessary to analyze it. Several methods of identification have been proposed, including:
(1) encoding, i.e., the attachment to each member of an identifier moiety which is more readily identified than the member proper. This has the disadvantage that the tag may itself influence the activity of the conjugate.
(2) spatial addressing, e.g., each member is synthesized only at a particular coordinate on or in a matrix, or in a particular chamber. This might be, for example, the location of a particular pin, or a particular well on a microtiter plate, or inside a “tea bag”.
The present invention is not limited to any particular form of identification.

However, it is possible to simply characterize those members of the library which are found to be active, based on the characteristic spectroscopic indicia of the various building blocks.

Solid phase synthesis permits greater control over which derivatives are formed. However, the solid phase could interfere with activity. To overcome this problem, some or all of the molecules of each member could be liberated, after synthesis but before screening.

Examples of candidate simple libraries which might be evaluated include derivatives of the following:

Cyclic Compounds Containing One Hetero Atom
Heteronitrogen
   pyrroles
      pentasubstituted pyrroles
   pyrrolidines
   pyrrolines
   prolines
   indoles
   beta-carbolines
   pyridines
      dihydropyridines
      1,4-dihydropyridines
      pyrido[2,3d]pyrimidines
      tetrahydro-3H-imidazo[4, 5-c] pyridines
   Isoquinolines
      tetrahydroisoquinolines
   quinolones
   beta-lactams
      azabicyclo[4.3.0]nonen-8-one amino acid Heterooxygen
   furans
      tetrahydrofurans
         2, 5-disubstituted tetrahydrofurans
   pyrans
      hydroxypyranones
      tetrahydroxypyranones
   gamma-butyrolactones
Heterosulfur
   sulfolenes

Cyclic Compounds with Two or More Hetero atoms
Multiple heteronitrogens
   imidazoles
   pyrazoles
   piperazines
      diketopiperazines
      arylpiperazines
      benzylpiperazines
   benzodiazepines
   1, 4-benzodiazepine-2, 5-diones
   hydantoins
      5-alkoxyhydantoins
   dihydropyrimidines
   1, 3-disubstituted-5, 6-dihydopyrimidine-2,4-diones
   cyclic ureas
   cyclic thioureas
   quinazolines
      chiral 3-substituted-quinazoline-2, 4-diones
   triazoles
      1,2,3-triazoles
   purines
Heteronitrogen and Heterooxygen
   dikelomorpholines
   isoxazoles
   isoxazolines
Heteronitrogen and Heterosulfur
   thiazolidines
      N-axylthiazolidines dihydrothiazoles
      2-methylene-2, 3-dihydrothiazates
      2-aminothiazoles
   thiophenes
      3-amino thiophenes
   4-thiazolidinones
   4-melathiazanones
   benzisothiazolones

For details on synthesis of libraries, see Nefzi, et al., Chem. Rev., 97:449-72 (1997), and references cited therein.

Pharmaceutical Methods and Preparations

The preferred animal subject of the present invention is a mammal. By the term “mammal” is meant an individual belonging to the class Mammalia. The invention is particularly useful in the treatment of human subjects, although it is intended for veterinary and nutritional uses as well. Preferred nonhuman subjects are of the orders Primata (e.g., apes and monkeys), Artiodactyla or Perissodactyla (e.g., cows, pigs, sheep, horses, goats), Carnivora (e.g., cats, dogs), Rodenta (e.g., rats, mice, guinea pigs, hamsters), Lagomorpha (e.g., rabbits) or other pet, farm or laboratory mammals.

The term “protection”, as used herein, is intended to include “prevention,” “suppression” and “treatment.” “Prevention”, strictly speaking, involves administration of the pharmaceutical prior to the induction of the disease (or other adverse clinical condition). “Suppression” involves administration of the composition prior to the clinical appearance of the disease. “Treatment” involves administration of the protective composition after the appearance of the disease.

It will be understood that in human and veterinary medicine, it is not always possible to distinguish between “preventing” and “suppressing” since the ultimate inductive event or events may be unknown, latent, or the patient is not ascertained until well after the occurrence of the event or events. Therefore, unless qualified, the term “prevention” will be understood to refer to both prevention in the strict sense, and to suppression.

The preventative or prophylactic use of a pharmaceutical involves identifying subjects who are at higher risk than the general population of contracting the disease, and administering the pharmaceutical to them in advance of the clinical appearance of the disease. The effectiveness of such use is measured by comparing the subsequent incidence or severity of the disease, or of particular symptoms of the disease, in the treated subjects against that in untreated subjects of the same high risk group.

While high risk factors vary from disease to disease, in general, these include (1) prior occurrence of the disease in one or more members of the same family, or, in the case of a contagious disease, in individuals with whom the subject has come into potentially contagious contact at a time when the earlier victim was likely to be contagious, (2) a prior occurrence of the disease in the subject, (3) prior occurrence of a related disease, or a condition known to increase the likelihood of the disease, in the subject; (4) appearance of a suspicious level of a marker of the disease, or a related disease or condition; (5) a subject who is immunologically compromised, e.g., by radiation treatment, HIV infection, drug use, etc., or (6) membership in a particular group (e.g., a particular age, sex, race, ethnic group, etc.) which has been epidemiologically associated with that disease.

A prophylaxis or treatment may be curative, that is, directed at the underlying cause of a disease, or ameliorative, that is, directed at the symptoms of the disease, especially those which reduce the quality of life.

It should also be understood that to be useful, the protection provided need not be absolute, provided that it is sufficient to carry clinical value. An agent which provides protection to a lesser degree than do competitive agents may still be of value if the other agents are ineffective for a particular individual, if it can be used in combination with other agents to enhance the level of protection, or if it is safer than competitive agents. It is desirable that there be a statistically significant (p=0.05 or less) improvement in the treated subject relative to an appropriate untreated control, and it is desirable that this improvement be at least 10%, more preferably at least 25%, still more preferably at least 50%, even more preferably at least 100%, in some indicia of the incidence or severity of the disease or of at least one symptom of the disease.

At least one of the drugs of the present invention may be administered, by any means that achieve their intended purpose, to protect a subject against a disease or other adverse condition. The form of administration may be systemic or topical. For example, administration of such a composition may be by various parenteral routes such as subcutaneous, intravenous, intradermal, intramuscular, intraperitoneal, intranasal, transdermal, or buccal routes. Alternatively, or concurrently, administration may be by the oral route. Parenteral administration can be by bolus injection or by gradual perfusion over time.

A typical regimen comprises administration of an effective amount of the drug, administered over a period ranging from a single dose, to dosing over a period of hours, days, weeks, months, or years.

It is understood that the suitable dosage of a drug of the present invention will be dependent upon the age, sex, health, and weight of the recipient, kind of concurrent treatment, if any, frequency of treatment, and the nature of the effect desired. However, the most preferred dosage can be tailored to the individual subject, as is understood and determinable by one of skill in the art, without undue experimentation. This will typically involve adjustment of a standard dose, e.g., reduction of the dose if the patient has a low body weight.

Prior to use in humans, a drug will first be evaluated for safety and efficacy in laboratory animals. In human clinical studies, one would begin with a dose expected to be safe in humans, based on the preclinical data for the drug in question, and on customary doses for analogous drugs (if any). If this dose is effective, the dosage may be decreased, to determine the minimum effective dose, if desired. If this dose is ineffective, it will be cautiously increased, with the patients monitored for signs of side effects. See, e.g., Berkow et al, eds., The Merck Manual, 15th edition, Merck and Co., Rahway, N.J., 1987; Goodman et al., eds., Goodman and Gilman's The Pharmacological Basis of Therapeutics, 8th edition, Pergamon Press, Inc., Elmsford, N.Y., (1990); Avery's Drug Treatment: Principles and Practice of Clinical Pharmacology and Therapeutics, 3rd edition, ADIS Press, LTD., Williams and Wilkins, Baltimore, MD. (1987), Ebadi, Pharmacology, Little, Brown and Co., Boston, (1985), which references and references cited therein, are entirely incorporated herein by reference.

The total dose required for each treatment may be administered by multiple doses or in a single dose. The protein may be administered alone or in conjunction with other therapeutics directed to the disease or directed to other symptoms thereof.

The appropriate dosage form will depend on the disease, the pharmaceutical, and the mode of administration; possibilities include tablets, capsules, lozenges, dental pastes, suppositories, inhalants, solutions, ointments and parenteral depots. See, e.g., Berker, supra, Goodman, supra, Avery, supra and Ebadi, supra, which are entirely incorporated herein by reference, including all references cited therein.

In the case of peptide drugs, the drug may be administered in the form of an expression vector comprising a nucleic acid encoding the peptide; such a vector, after incorporation into the genetic complement of a cell of the patient, directs synthesis of the peptide. Suitable vectors include genetically engineered poxviruses (vaccinia), adenoviruses, adeno-associated viruses, herpesviruses and lentiviruses which are or have been rendered nonpathogenic.

In addition to at least one drug as described herein, a pharmaceutical composition may contain suitable pharmaceutically acceptable carriers, such as excipients, carriers and/or auxiliaries which facilitate processing of the active compounds into preparations which can be used pharmaceutically. See, e.g., Berker, supra, Goodman, supra, Avery, supra and Ebadi, supra, which are entirely incorporated herein by reference, included all references cited therein.

Assay Compositions and Methods

Target Organism

The invention contemplates that it may be appropriate to ascertain or to mediate the biological activity of a substance of this invention in a target organism.

The target organism may be a plant, animal, or microorganism.

In the case of a plant, it may be an economic plant, in which case the drug may be intended to increase the disease, weather or pest resistance, alter the growth characteristics, or otherwise improve the useful characteristics or mute undesirable characteristics of the plant. Or it may be a weed, in which case the drug may be intended to kill or otherwise inhibit the growth of the plant, or to alter its characteristics to convert it from a weed to an economic plant. The plant may be a tree, shrub, crop, grass, etc. The plant may be an algae (which are in some cases also microorganisms), or a vascular plant, especially gymnosperms (particularly conifers) and angiosperms. Angiosperms may be monocots or dicots. The plants of greatest interest are rice, wheat, corn, alfalfa, soybeans, potatoes, peanuts, tomatoes, melons, apples, pears, plums, pineapples, fir, spruce, pine, cedar, and oak.

If the target organism is a microorganism, it may be electrodes in the chip were used to create electrokinetic forces capable of driving molecules through these micro-channels to perform electrophoretic separations. Ribosomal peaks were measured by fluorescence signal and displayed in an electropherogram. A successful total RNA sample featured 2 distinct ribosomal peaks (18S and 28S rRNA).

Biotinylated cRNA Hybridization Target.

Total RNA was prepared for use as a hybridization target as described in the manufacturer's instructions for CodeLink Expression Bioarrays(™) (Amersham Biosciences). The CodeLink Expression Bioarrays utilize nucleic acid hybridization of a biotin-labeled complementary RNA(cRNA) target with DNA oligonucleotide probes attached to a gel matrix.

The biotin-labeled cRNA target is prepared by a linear amplification method. Poly (A) +RNA (within the total RNA population) is primed for reverse transcription by a DNA oligonucleotide containing a T7 RNA polymerase promoter 5′ to a (dT) 24 sequence. After second-strand cDNA synthesis, the cDNA serves as the template in an in vitro transcription (IVT) reaction to produce the target cRNA. The IVT is performed in the presence of biotinylated nucleotides to label the target cRNA. This procedure results in a 50-200 fold linear amplification of the input poly (A) +RNA.

Hybridization Probes.

The oligonucleotide probes were provided by the Codelink Uniset Mouse I Bioarray (Amersham, product code 300013). Amine-terminated oligonucleotide probes are attached to a three-dimensional polyacrylamide gel matrix. There are 10,000 oligonucleotide probes, each specific to a well-characterized mouse gene.Each mouse gene is representative of a unique gene cluster from the fourth quarter 2001 Genbank Unigene build. There are also 500 control probes.

The sequences of the probes is proprietary to Amersham. However, for each probe, Amersham identifies the corresponding mouse gene by NCBI accession number, OGS, LocusLink, Unigene Cluster ID, and description (name). This information should be available from Amersham. In the case of the differentially expressed probes, this information is duplicated in master table 1. For the complete list, see http://www4.amershambiosciences.com/aptrix/upp01077.nsf/Cont ent/codelink_literature

Under “Gene Lists”, select “Uniset Human I”, and a gene list, in Excel format, can be downloaded.

Hybridization

Using the cRNA target, the hybridization reaction mixture is prepared and loaded until array chambers for bioarray processing as set forth in the manufacturer's instructions for CodeLink Gene Expression Bioarrays™ (Amerhsam Biosciences). Each sample is hybridized to an individual microarray. Hybridization is at 37° C. The hybridization buffer is prepared as set forth in the Motorola instructions. Hybridization to the microarray is detected with an avidinated fluorescent reagent, Streptavidin-Alexa Fluor ®647 (Amersham).

Mouse Gene Expression Analysis

Processed arrays were scanned using a GenePix 4000B Microarray Scanner (Axon Instruments, Inc.); array images were acquired using the Amersham CodeLink™ Analysis Software (Release 2.2). The Amersham CodeLink™ Analysis Software gives an integrated optical density (IOD) value for every spot; a unique background value for that spot is subtracted, resulting in “raw” data points. Individual chips are then normalized by the Amersham Codelink™ software according to the median raw intensity for all 10,000 genes. A negative control threshold is also calculated according to the control probes. A significant difference in expression between samples was defined as a minimum of 2-fold change in expression values. Genes with expression values below the negative control threshold were eliminated from the analysis and then the expression data was analyzed to identify genes whose expression levels changed significantly with respect to:
Normal mice compared to hyperinsulinemic mice at 2, 4, 8 and 16 weeks, and 6 months, on normal vs. high-fat diet.
Normal mice compared to hyperinsulinemic/hyperglycemic mice at 2, 4, 8 and 16 weeks, and 6 months, on normal vs. high-fat diet.
Hyperinsulinemic compared to hyperinsulinemic/hyperglycemic mice at 2, 4, 8 and 16 weeks, and 6 months, on high-fat diets.

Database Searches Nucleotide sequences and predicted amino acid sequences were compared to public domain databases using the Blast 2.0 program (National Center for Biotechnology Information, National Institutes of Health). Nucleotide sequences were displayed using ABI prism Edit View 1.0.1 (PE Applied Biosystems, Foster City, Calif.).

Nucleotide database searches were conducted with the then current version of BLASTN 2.0.12, see Altschul, et al., “Gapped BLAST and PSI-BLAST: a new generation of protein database search programs”, Nucleic Acids Res., 25:3389-3402 (1997). Searches employed the default parameters, unless otherwise stated.

For blastN searches, the default was the blastN matrix (1−, 3), with gap penalties of 5 for existence and 2 for extension.

Protein database searches were conducted with the then-current version of BLAST X, see Altschul et al. (1997), supra. Searches employed the default parameters, unless otherwise stated. The scoring matrix was BLOSUM62, with gap costs of 11 for existence and 1 for extension. The standard low complexity filter was used. “ref” indicates that NCBI's RefSeq is the source database. The identifier that follows is a RefSeq accession number, not a GenBank accession number. “RefSeq sequences are derived from GenBank and provide non-redundant curated data representing our current knowledge of known genes. Some records include additional sequence information that was never submitted to an archival database but is available in the literature. A small number of sequences are provided through collaboration; the underlying primary sequence data is available in GenBank, but may not be available in any one GenBank record. RefSeq sequences are not submitted primary sequences. RefSeq records are owned by NCBI and therefore can be updated as needed to maintain current annotation or to incorporate additional sequence information.” See also http://www.ncbi.nlm.nih.gov/LocusLink/refseq.html

It will be appreciated by those in the art that the exact results of a database search will change from day to day, as new sequences are added. Also, if you query with a longer version of the original sequence, the results will change. The results given here were obtained at one time and no guarantee is made that the exact same hits would be obtained in a search on the filing date. However, if an alignment between a particular query sequence and a particular database sequence is discussed, that alignment should not change (if the parameters and sequences remain unchanged).

Northern Analysis.

Northern analysis may be used to confirm the results. Favorable and unfavorable genes, identified as described above, or fragments thereof, will be used as probes in Northern hybridization analyses to confirm their differential expression. Total RNA isolated from Control, Hyperinsulinemic and Type-II Diabetic mice will be resolved by agarose gel electrophoresis through a 1% agarose, 1% formaldehyde denaturing gel, transferred to positively charged nylon membrane, and hybridized to a probe labeled with [32P] dCTP that was generated from the aforementioned gene or fragment using the Random Primed DNA Labeling Kit (Roche, Palo Alto, Calif.).

Real-Time RNA Analysis

Real-time RNA analysis may also be used for confirmation. For “real-time” RNA analysis, RNA will be converted to cDNA and then probed with gene-specific primers made for each clone. “Real-time” incorporation of fluorescent dye will be measured to determine the amount of specific transcript present in each sample. Sample differences (control vs. hyperinsulinemic, hyperinsulinemic vs. diabetic, or control vs. diabetic) of 2-fold or greater (in either direction) will be considered differentially expressed. Confirmation using several independent animals is desirable.

In situ Hybridization

Another form of confirmation may be provided by nonisotopic in situ hybridizations (NISH) on selected human (obtained by Tissue Informatics) and mouse tissues using cRNA probes generated from mouse genes found to be up-or down-regulated during the disease progression. Nonisotopic in situ hybridizations may also be performed on mouse tissues using cRNA probes generated from all “novel” cDNA's identified through PCR subtractive hybridizations. These cRNA's will hybridize to their corresponding messenger RNA's present in cells and will provide information regarding the particular cell types within a tissue that is expressing the particular gene as well as the relative level of gene expression. The cRNA probes may be generated by in vitro transcription of template cDNA by Sp6 or T7 RNA polymerase in the presence of digoxigenin-11-UTP (Roche Molecular Biochemicals, Mannheim, Germany; Pardue, M. L. 1985. In: In situ hybridization, Nucleic acid hybridization, a practical approach: IRL Press, Oxford, 179-202).

Transgenic Animals

Transgenic expression may be used to confirm the results. In one embodiment, a mouse is engineered to overexpress the favorable or unfavorable mouse gene in question. In another embodiment, a mouse is engineered to express the corresponding favorable or unfavorable human gene. In a third embodiment, a nonhuman animal other than a mouse, such as a rat, rabbit, goat, sheep or pig, is engineered to express the favorable or unfavorable mouse or human gene.

Hyperquantitative Tissue Analysis

In addition to gene expression analysis the liver sections can also be analyzed using TissueInformatics, Inc's TissueAnalytics™ software. A single representative section may be cut from each liver block, placed on a slide, and stained with H&E. Digital images of each slide may be acquired using an research microscope and digital camera (Olympus E600 microscope and Sony DKC-ST5). These images were acquired at 20×magnification with a resolution of 0.64 mm/pixel. A hyperquantitative analysis may be performed on the resulting images: First a digital image analysis can identify and annotate structural objects in a tissue using machine vision. These objects, that are constituents of the tissue, can be annotated because they are visually identifiable and have a biological meaning like hepatocytes, sinusoids, vacuoles. Subsequently a quantification of these structures regarding their geometric properties like area or stain intensities and their relationship to the field of view or per unit area in terms of a % coverage may be performed. Features or parameters for hyper-quantification are specific for each tissue, and may also include relations between features, measures of overall heterogeneity, including orientation, relative locations, and textures.

Correlation Analysis

Mathematical statistics provides a rich set of additional tools to analyze time resolved data sets of hyper-quantitative and gene expression profiles for similarities, including rank correlation, the calculation of regression and correlations coefficients, and clustering. Continuous functions may also be fitted through the data points of individual gene and tissue feature data. Relation between gene expression and hyper-quantitative tissue data may be linear or non-linear, in synchronous or asynchronous arrangements.

A Spearman rank correlation analysis using was done on the 2 classes of measurements (Genes and Tissues Features) to help identify other significant genes. A small number of genes that did not meet the 2-Fold difference for significance were added to the list of genes based on their correlation with tissue features.

Citation of documents herein is not intended as an admission that any of the documents cited herein is pertinent prior art, or an admission that the cited documents is considered material to the patentability of any of the claims of the present application. All statements as to the date or representation as to the contents of these documents is based on the information available to the applicant and does not constitute any admission as to the correctness of the dates or contents of these documents.

The appended claims are to be treated as a non-limiting recitation of preferred embodiments.

In addition to those set forth elsewhere, the following references are hereby incorporated by reference, in their most recent editions as of the time of filing of this application: Kay, Phage Display of Peptides and Proteins: A Laboratory Manual; the John Wiley and Sons Current Protocols series, including Ausubel, Current Protocols in Molecular Biology; Coligan, Current Protocols in Protein Science; Coligan, Current Protocols in Immunology; Current Protocols in Human Genetics; Current Protocols in Cytometry; Current Protocols in Pharmacology; Current Protocols in Neuroscience; Current Protocols in Cell Biology; Current Protocols in Toxicology; Current Protocols in Field Analytical Chemistry; Current Protocols in Nucleic Acid Chemistry; and Current Protocols in Human Genetics; and the following Cold Spring Harbor Laboratory publications: Sambrook, Molecular Cloning: A Laboratory Manual; Harlow, Antibodies: A Laboratory Manual; Manipulating the Mouse Embryo: A Laboratory Manual; Methods in Yeast Genetics: A Cold Spring Harbor Laboratory Course Manual; Drosophila Protocols; Imaging Neurons: A Laboratory Manual; Early Development of Xenopus laevis: A Laboratory Manual; Using Antibodies: A Laboratory Manual; At the Bench: A Laboratory Navigator; Cells: A Laboratory Manual; Methods in Yeast Genetics: A Laboratory Course Manual; Discovering Neurons: The Experimental Basis of Neuroscience; Genome Analysis: A Laboratory Manual Series ; Laboratory DNA Science; Strategies for Protein Purification and Characterization: A Laboratory Course Manual; Genetic Analysis of Pathogenic Bacteria: A Laboratory Manual; PCR Primer: A Laboratory Manual; Methods in Plant Molecular Biology: A Laboratory Course Manual ; Manipulating the Mouse Embryo: A Laboratory Manual; Molecular Probes of the Nervous System; Experiments with Fission Yeast: A Laboratory Course Manual; A Short Course in Bacterial Genetics: A Laboratory Manual and Handbook for Escherichia coli and Related Bacteria; DNA Science: A First Course in Recombinant DNA Technology; Methods in Yeast Genetics: A Laboratory Course Manual; Molecular Biology of Plants: A Laboratory Course Manual.

All references cited herein, including journal articles or abstracts, published, corresponding, prior or otherwise related U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by reference.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Any description of a class or range as being useful or preferred in the practice of the invention shall be deemed a description of any subclass (e.g., a disclosed class with one or more disclosed members omitted) or subrange contained therein, as well as a separate description of each individual member or value in said class or range.

The description of preferred embodiments individually shall be deemed a description of any possible combination of such preferred embodiments, except for combinations which are impossible (e.g, mutually exclusive choices for an element of the invention) or which are expressly excluded by this specification.

If an embodiment of this invention is disclosed in the prior art, the description of the invention shall be deemed to include the invention as herein disclosed with such embodiment excised.

Introduction to Master Tables

The master tables reflect applicants' analysis of the gene chip data.

For each probe corresponding to a differentially expressed mouse gene, Master Table 1 identifies
Col. 1: The mouse gene (upper) and mouse protein (lower) database accession #s.
Col. 2: The corresponding mouse Unigene Cluster, as of the 4^{th} Quarter 2001 build.
Col. 3: The behavior (differential expression) observed for the mouse gene. This column identifies the gene as favorable(F) or unfavorable (U) on the basis of its differential behavior. There are three possible comparisons, HI-D, C-HI, and C-D, where C=control (normal), HI=hyperinsulinemic, and D=diabetic.
If the level of the gene in the former state is at least two-fold that in the latter state, it is considered unfavorable. If the level of the gene in the former state is not more than half (i.e., not more than negative two fold) that in the latter state, it is considered favorable.
Col. 4: A related human protein, identified by its database accession number. Usually, several such proteins are identified relative to each mouse gene. These proteins have been identified by BLAST searches, as explained in cols. 6-8.
Col. 5: The name of the related human protein.
Col. 6: The score (in bits) for the alignment performed by the BLAST program.
Col. 7: The E-value for the alignment performed by the BLAST program. It is worth noting that Unigene considers a Blastx E Value of less than 1e-6 to be a “match” to the reference sequence of a cluster.
Col. 8: The BLAST search strategy used. MG indicates that the mouse gene was used as the query sequence in a BlastX search. MP means that the mouse protein was used as the query sequence in a BlastP search. HGP means that first the mouse gene was used in a BlastN search for a human gene, and then the human gene was used in a BLASTX search for the human protein.

Master Table 1 is divided into three subtables on the basis of the Behavior” in col. 3. If a gene has at least one favorable behavior, and no unfavorable ones, it is put into Subtable 1A. In the opposite case, it is put into Subtable 1B. If its behavior is mixed, i.e., at least one favorable and at least one unfavorable, it is put into Subtable 1C.

Master Table 2 has just three columns.
Col. 1: Mouse gene.
Col. 2: behavior. Same as col. 3 in Master table 1.
Col. 3: Human protein classes. Based on the related human proteins defined in Master Table 1, Master Table 2 generalizes, if possible as to classes of human proteins which are expected to have similar behavior. For a given mouse gene, several human protein classes may be listed because of the diversity of the human proteins found to be related. In some cases, the stated human protein classes may be hierarchial, e.g., one may be a subset of another. In other cases, the stated classes may be non-overlapping but related. And in yet other cases, the stated classes may be algae, bacteria, fungi, or a virus (although the biological activity of a virus must be determined in a virus-infected cell). The microorganism may be human or other animal or plant pathogen, or it may be nonpathogenic. It may be a soil or water organism, or one which normally lives inside other living things.

If the target organism is an animal, it may be a vertebrate or a nonvertebrate animal. Nonvertebrate animals are chiefly of interest when they act as pathogens or parasites, and the drugs are intended to act as biocidic or biostatic agents. Nonvertebrate animals of interest include worms, mollusks, and arthropods.

The target organism may also be a vertebrate animal, i.e., a mammal, bird, reptile, fish or amphibian. Among mammals, the target animal preferably belongs to the order Primata (humans, apes and monkeys), Artiodactyla (e.g., cows, pigs, sheep, goats, horses), Rodenta (e.g., mice, rats) Lagomorpha (e.g., rabbits, hares), or Carnivora (e.g., cats, dogs). Among birds, the target animals are preferably of the orders Anseriformes (e.g., ducks, geese, swans) or Galliformes (e.g., quails, grouse, pheasants, turkeys and chickens). Among fish, the target animal is preferably of the order Clupeiformes (e.g., sardines, shad, anchovies, whitefish, salmon).

Target Tissues

The term “target tissue” refers to any whole animal, physiological system, whole organ, part of organ, miscellaneous tissue, cell, or cell component (e.g., the cell membrane) of a target animal in which biological activity may be measured.

Routinely in mammals one would choose to compare and contrast the biological impact on virtually any and all tissues which express the subject receptor protein. The main tissues to use are: brain, heart, lung, kidney, liver, pancreas, skin, intestines, adipose, stomach, skeletal muscle, adrenal glands, breast, prostate, vasculature, retina, cornea, thyroid gland, parathyroid glands, thymus, bone marrow, bone, etc.

Another classification would be by cell type: B cells, T cells, macrophages, neutrophils, eosinophils, mast cells, platelets, megakaryocytes, erythrocytes, bone marrow stomal cells, fibroblasts, neurons, astrocytes, neuroglia, microglia, epithelial cells (from any organ, e.g. skin, breast, prostate, lung, intestines etc), cardiac muscle cells, smooth muscle cells, striated muscle cells, osteoblasts, osteocytes, chondroblasts, chondrocytes, keratinocytes, melanocytes, etc.

Of course, in the case of a unicellular organism, there is no distinction between the “target organism” and the “target tissue”.

Screening Assays

Assays intended to determine the binding or the biological activity of a substance are called preliminary screening assays.

Screening assays will typically be either in vitro (cell-free) assays (for binding to an immobilized receptor) or cell-based assays (for alterations in the phenotype of the cell). They will not involve screening of whole multicellular organisms, or isolated organs. The comments on diagnostic biological assays apply mutatis mutandis to screening cell-based assays.

In Vitro vs. In Vivo Assays

The term in vivo is descriptive of an event, such as binding or enzymatic action, which occurs within a living organism. The organism in question may, however, be genetically modified. The term in vitro refers to an event which occurs outside a living organism. Parts of an organism (e.g., a membrane, or an isolated biochemical) are used, together with artificial substrates and/or conditions. For the purpose of the present invention, the term in vitro excludes events occurring inside or on an intact cell, whether of a unicellular or multicellular organism.

In vivo assays include both cell-based assays, and organismic assays. The cell-based assays include both assays on unicellular organisms, and assays on isolated cells or cell cultures derived from multicellular organisms. The cell cultures may be mixed, provided that they are not organized into tissues or organs. The term organismic assay refers to assays on whole multicellular organisms, and assays on isolated organs or tissues of such organisms.

In vitro Diagnostic Methods and Reagents

The in vitro assays of the present invention may be applied to any suitable analyte-containing sample, and may be qualitative or quantitative in nature.

Sample

The sample will normally be a biological fluid, such as blood, urine, lymph, semen, milk, or cerebrospinal fluid, or a fraction or derivative thereof, or a biological tissue, in the form of, e.g., a tissue section or homogenate. However, the sample conceivably could be (or derived from) a food or beverage, a pharmaceutical or diagnostic composition, soil, or surface or ground water. If a biological fluid or tissue, it may be taken from a human or other mammal, vertebrate or animal, or from a plant. The preferred sample is blood, or a fraction or derivative thereof.

Binding and Reaction Assays

The assay may be a binding assay, in which one step involves the binding of a diagnostic reagent to the analyte, or a reaction assay, which involves the reaction of a reagent with the analyte. The reagents used in a binding assay may be classified as to the nature of their interaction with analyte: (1) analyte analogues, or (2) analyte binding molecules (ABM). They may be labeled or insolubilized.

In a reaction assay, the assay may look for a direct reaction between the analyte and a reagent which is reactive with the analyte, or if the analyte is an enzyme or enzyme inhibitor, for a reaction catalyzed or inhibited by the analyte. The reagent may be a reactant, a catalyst, or an inhibitor for the reaction.

An assay may involve a cascade of steps in which the product of one step acts as the target for the next step. These steps may be binding steps, reaction steps, or a combination thereof.

Signal Producing System (SPS)

In order to detect the presence, or measure the amount, of an analyte, the assay must provide for a signal producing system (SPS) in which there is a detectable difference in the signal produced, depending on whether the analyte is present or absent (or, in a quantitative assay, on the amount of the analyte). The detectable signal may be one which is visually detectable, or one detectable only with instruments. Possible signals include production of colored or luminescent products, alteration of the characteristics (including amplitude or polarization) of absorption or emission of radiation by an assay component or product, and precipitation or agglutination of a component or product. The term “signal” is intended to include the discontinuance of an existing signal, or a change in the rate of change of an observable parameter, rather than a change in its absolute value. The signal may be monitored manually or automatically.

In a reaction assay, the signal is often a product of the reaction. In a binding assay, it is normally provided by a label borne by a labeled reagent.

Labels

The component of the signal producing system which is most intimately associated with the diagnostic reagent is called the “label”. A label may be, e.g., a radioisotope, a fluorophore, an enzyme, a co-enzyme, an enzyme substrate, an electron-dense compound, an agglutinable particle.

The radioactive isotope can be detected by such means as the use of a gamma counter or a scintillation counter or by autoradiography. Isotopes which are particularly useful for the purpose of the present invention include ³H, ¹²⁵I, ¹³¹T, ³⁵S, ¹⁴C, ³²P and ³³P ¹²⁵I is preferred for antibody labeling.

The label may also be a fluorophore. When the fluorescently labeled reagent is exposed to light of the proper wave length, its presence can then be detected due to fluorescence. Among the most commonly used fluorescent labeling compounds are fluorescein isothiocyanate, rhodamine, phycoerythrin, phycocyanin, allophycocyanin, o-phthaldehyde and fluorescamine.

Alternatively, fluorescence-emitting metals such as ¹²⁵Eu, or others of the lanthanide series, may be incorporated into a diagnostic reagent using such metal chelating groups as diethylenetriaminepentaacetic acid (DTPA) of ethylenediamine-tetraacetic acid (EDTA).

The label may also be a chemiluminescent compound. The presence of the chemiluminescently labeled reagent is then determined by detecting the presence of luminescence that arises during the course of a chemical reaction. Examples of particularly useful chemiluminescent labeling compounds are luminol, isolumino, theromatic acridinium ester, imidazole, acridinium salt and oxalate ester.

Likewise, a bioluminescent compound may be used for labeling. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labeling are luciferin, luciferase and aequorin.

Enzyme labels, such as horseradish peroxidase and alkaline phosphatase, are preferred. When an enzyme label is used, the signal producing system must also include a substrate for the enzyme. If the enzymatic reaction product is not itself detectable, the SPS will include one or more additional reactants so that a detectable product appears.

An enzyme analyte may act as its own label if an enzyme inhibitor is used as a diagnostic reagent.

Binding Assay Formats

Binding assays may be divided into two basic types, heterogeneous and homogeneous. In heterogeneous assays, the interaction between the affinity molecule and the analyte does not affect the label, hence, to determine the amount or presence of analyte, bound label must be separated from free label. In homogeneous assays, the interaction does affect the activity of the label, and therefore analyte levels can be deduced without the need for a separation step.

In one embodiment, the ABM is insolubilized by coupling it to a macromolecular support, and analyte in the sample is allowed to compete with a known quantity of a labeled or specifically labelable analyte analogue. The “analyte analogue” is a molecule capable of competing with analyte for binding to the ABM, and the term is intended to include analyte itself. It may be labeled already, or it may be labeled subsequently by specifically binding the label to a moiety differentiating the analyte analogue from analyte. The solid and liquid phases are separated, and the labeled analyte analogue in one phase is quantified. The higher the level of analyte analogue in the solid phase, i.e., sticking to the ABM, the lower the level of analyte in the sample.

In a “sandwich assay”, both an insolubilized ABM, and a labeled ABM are employed. The analyte is captured by the insolubilized ABM and is tagged by the labeled ABM, forming a ternary complex. The reagents may be added to the sample in either order, or simultaneously. The ABMs may be the same or different. The amount of labeled ABM in the ternary complex is directly proportional to the amount of analyte in the sample.

The two embodiments described above are both heterogeneous assays. However, homogeneous assays are conceivable. The key is that the label be affected by whether or not the complex is formed.

Conjugation Methods

A label may be conjugated, directly or indirectly (e.g., through a labeled anti-ABM antibody), covalently (e.g., with SPDP) or noncovalently, to the ABM, to produce a diagnostic reagent. Similarly, the ABM may be conjugated to a solid phase support to form a solid phase (“capture”) diagnostic reagent.

Suitable supports include glass, polystyrene, polypropylene, polyethylene, dextran, nylon, amylases, natural and modified celluloses, polyacrylamides, agaroses, and magnetite. The nature of the carrier can be either soluble to some extent or insoluble for the purposes of the present invention.

The support material may have virtually any possible structural configuration so long as the coupled molecule is capable of binding to its target. Thus the support configuration may be spherical, as in a bead, or cylindrical, as in the inside surface of a test tube, or the external surface of a rod. Alternatively, the surface may be flat such as a sheet, test strip, etc.

Biological Assays

A biological assay measures or detects a biological response of a biological entity to a substance.

The biological entity may be a whole organism, an isolated organ or tissue, freshly isolated cells, an immortalized cell line, or a subcellular component (such as a membrane; this term should not be construed as including an isolated receptor). The entity may be, or may be derived from, an organism which occurs in nature, or which is modified in some way. Modifications may be genetic (including radiation and chemical mutants, and genetic engineering) or somatic (e.g., surgical, chemical, etc.). In the case of a multicellular entity, the modifications may affect some or all cells. The entity need not be the target organism, or a derivative thereof, if there is a reasonable correlation between bioassay activity in the assay entity and biological activity in the target organism.

The entity is placed in a particular environment, which may be more or less natural. For example, a culture medium may, but need not, contain serum or serum substitutes, and it may, but need not, include a support matrix of some kind, it may be still, or agitated. It may contain particular biological or chemical agents, or have particular physical parameters (e.g., temperature), that are intended to nourish or challenge the biological entity.

There must also be a detectable biological marker for the response. At the cellular level, the most common markers are cell survival and proliferation, cell behavior (clustering, motility), cell morphology (shape, color), and biochemical activity (overall DNA synthesis, overall protein synthesis, and specific metabolic activities, such as utilization of particular nutrients, e.g., consumption of oxygen, production of CO₂, production of organic acids, uptake or discharge of ions).

The direct signal produced by the biological marker may be transformed by a signal producing system into a different signal which is more observable, for example, a fluorescent or colorimetric signal.

The entity, environment, marker and signal producing system are chosen to achieve a clinically acceptable level of sensitivity, specificity and accuracy.

In some cases, the goal will be to identify substances which mediate the biological activity of a natural biological entity, and the assay is carried out directly with that entity. In other cases, the biological entity is used simply as a model of some more complex (or otherwise inconvenient to work with) biological entity. In that event, the model biological entity is used because activity in the model system is considered more predictive of activity in the ultimate natural biological entity than is simple binding activity in an in vitro system. The model entity is used instead of the ultimate entity because the former is more expensive or slower to work with, or because ethical considerations forbid working with the ultimate entity yet.

The model entity may be naturally occurring, if the model entity usefully models the ultimate entity under some conditions. Or it may be non-naturally occurring, with modifications that increase its resemblance to the ultimate entity.

Transgenic animals, such as transgenic mice, rats, and rabbits, have been found useful as model systems.

In cell-based model assays, where the biological activity is mediated by binding to a receptor (target protein), the receptor may be functionally connected to a signal (biological marker) producing system, which may be endogenous or exogenous to the cell. There are a number of techniques of doing this.

“Zero-Hybrid” Systems

In these systems, the binding of a peptide to the target protein results in a screenable or selectable phenotypic change, without resort to fusing the target protein (or a ligand binding moiety thereof) to an endogenous protein. It may be that the target protein is endogenous to the host cell, or is substantially identical to an endogenous receptor so that it can take advantage of the latter's native signal transduction pathway. Or sufficient elements of the signal transduction pathway normally associated with the target protein may be engineered into the cell so that the cell signals binding to the target protein.

“One-Hybrid” Systems

In these systems, a chimera receptor, a hybrid of the target protein and an endogenous receptor, is used. The chimeric receptor has the ligand binding characteristics of the target protein and the signal transduction characteristics of the endogenous receptor. Thus, the normal signal transduction pathway of the endogenous receptor is subverted.

Preferably, the endogenous receptor is inactivated, or the conditions of the assay avoid activation of the endogenous receptor, to improve the signal-to-noise ratio.

See Fowlkes U.S. Pat. No. 5,789,184 for a yeast system.

Another type of “one-hybrid” system combines a peptide: DNA-binding domain fusion with an unfused target receptor that possesses an activation domain.

“Two-Hybrid” System

In a preferred embodiment, the cell-based assay is a two hybrid system. This term implies that the ligand is incorporated into a first hybrid protein, and the receptor into a second hybrid protein. The first hybrid also comprises component A of a signal generating system, and the second hybrid comprises component B of that system. Components A and B, by themselves, are insufficient to generate a signal. However, if the ligand binds the receptor, components A and B are brought into sufficiently close proximity so that they can cooperate to generate a signal.

Components A and B may naturally occur, or be substantially identical to moieties which naturally occur, as components of a single naturally occurring biomolecule, or they may naturally occur, or be substantially identical to moieties which naturally occur, as separate naturally occurring biomolecules which interact in nature.

Two-Hybrid System: Transcription Factor Type

In a preferred “two-hybrid” embodiment, one member of a peptide ligand:receptor binding pair is expressed as a fusion to a DNA-binding domain (DBD) from a transcription factor (this fusion protein is called the “bait”), and the other is expressed as a fusion to a transactivation domain (TAD) (this fusion protein is called the “fish”, the “prey”, or the “catch”). The transactivation domain should be complementary to the DNA-binding domain, i.e., it should interact with the latter so as to activate transcription of a specially designed reporter gene that carries a binding site for the DNA-binding domain. Naturally, the two fusion proteins must likewise be complementary.

This complementarity may be achieved by use of the complementary and separable DNA-binding and transcriptional activator domains of a single transcriptional activator protein, or one may use complementary domains derived from different proteins. The domains may be identical to the native domains, or mutants thereof. The assay members may be fused directly to the DBD or TAD, or fused through an intermediated linker.

The target DNA operator may be the native operator sequence, or a mutant operator. Mutations in the operator may be coordinated with mutations in the DBD and the TAD. An example of a suitable transcription activation system is one comprising the DNA-binding domain from the bacterial repressor LexA and the activation domain from the yeast transcription factor Gal4, with the reporter gene operably linked to the LexA operator.

It is not necessary to employ the intact target receptor; just the ligand-binding moiety is sufficient.

The two fusion proteins may be expressed from the same or different vectors. Likewise, the activatable reporter gene may be expressed from the same vector as either fusion protein (or both proteins), or from a third vector.

Potential DNA-binding domains include Gal4, LexA, and mutant domains substantially identical to the above.

Potential activation domains include E. coli B42, Gal4 activation domain II, and HSV VP16, and mutant domains substantially identical to the above.

Potential operators include the native operators for the desired activation domain, and mutant domains substantially identical to the native operator.

The fusion proteins may comprise nuclear localization signals.

The assay system will include a signal producing system, too. The first element of this system is a reporter gene operably linked to an operator responsive to the DBD and TAD of choice. The expression of this reporter gene will result, directly or indirectly, in a selectable or screenable phenotype (the signal). The signal producing system may include, besides the reporter gene, additional genetic or biochemical elements which cooperate in the production of the signal. Such an element could be, for example, a selective agent in the cell growth medium. There may be more than one signal producing system, and the system may include more than one reporter gene.

The sensitivity of the system may be adjusted by, e.g., use of competitive inhibitors of any step in the activation or signal production process, increasing or decreasing the number of operators, using a stronger or weaker DBD or TAD, etc.

When the signal is the death or survival of the cell in question, or proliferation or nonproliferation of the cell in question, the assay is said to be a selection. When the signal merely results in a detectable phenotype by which the signaling cell may be differentiated from the same cell in a nonsignaling state (either way being a living cell), the assay is a screen. However, the term “screening assay” may be used in a broader sense to include a selection. When the narrower sense is intended, we will use the term “nonselective screen”.

Various screening and selection systems are discussed in Ladner, U.S. Pat. No. 5,198,346.

Screening and selection may be for or against the peptide: target protein or compound:target protein interaction.

Preferred assay cells are microbial (bacterial, yeast, algal, protozooal), invertebrate, vertebrate (esp. mammalian, particularly human). The best developed two-hybrid assays are yeast and mammalian systems.

Normally, two hybrid assays are used to determine whether a protein X and a protein Y interact, by virtue of their ability to reconstitute the interaction of the DBD and the TAD. However, augmented two-hybrid assays have been used to detect interactions that depend on a third, non-protein ligand.

For more guidance on two-hybrid assays, see Brent and Finley, Jr., Ann. Rev. Genet., 31:663-704 (1997); Fremont-Racine, et al., Nature Genetics, 277-281 (Jul. 16, 1997); Allen, et al., TIBS, 511-16 (December. 1995); LeCrenier, et al., BioEssays, 20:1-6 (1998); Xu, et al., Proc. Nat. Acad. sci. (USA), 94:12473-8 (November. 1992); Esotak, et al., Mol. Cell. Biol., 15:5820-9 (1995); Yang, et al., Nucleic Acids Res., 23:1152-6 (1995); Bendixen, et al., Nucleic Acids Res., 22:1778-9 (1994); Fuller, et al., BioTechniques, 25:85-92 (July 1998); Cohen, et al., PNAS (USA) 95:14272-7 (1998); Kolonin and Finley, Jr., PNAS (USA) 95:14266-71 (1998). See also Vasavada, et al., PNAS (USA), 88:10686-90 (1991) (contingent replication assay), and Rehrauer, et al., J. Biol. Chem., 271:23865-73 91996) (LexA repressor cleavage assay).

Two-Hybrid Systems: Reporter Enzyme Type

In another embodiment, the components A and B reconstitute an enzyme which is not a transcription factor.

As in the last example, the effect of the reconstitution of the enzyme is a phenotypic change which may be a screenable change, a selectable change, or both.

In vivo Diagnostic Uses

Radio-labeled ABM may be administered to the human or animal subject. Administration is typically by injection, e.g., intravenous or arterial or other means of administration in a quantity sufficient to permit subsequent dynamic and/or static imaging using suitable radio-detecting devices. The dosage is the smallest amount capable of providing a diagnostically effective image, and may be determined by means conventional in the art, using known radio-imaging agents as a guide.

Typically, the imaging is carried out on the whole body of the subject, or on that portion of the body or organ relevant to the condition or disease under study. The amount of radio-labeled ABM accumulated at a given point in time in relevant target organs can then be quantified. A particularly suitable radio-detecting device is a scintillation camera, such as a gamma camera. A scintillation camera is a stationary device that can be used to image distribution of radio-labeled ABM. The detection device in the camera senses the radioactive decay, the distribution of which can be recorded. Data produced by the imaging system can be digitized. The digitized information can be analyzed over time discontinuously or continuously. The digitized data can be processed to produce images, called frames, of the pattern of uptake of the radio-labeled ABM in the target organ at a discrete point in time. In most continuous (dynamic) studies, quantitative data is obtained by observing changes in distributions of radioactive decay in target organs over time. In other words, a time-activity analysis of the data will illustrate uptake through clearance of the radio-labeled binding protein by the target organs with time.

Various factors should be taken into consideration in selecting an appropriate radioisotope. The radioisotope must be selected with a view to obtaining good quality resolution upon imaging, should be safe for diagnostic use in humans and animals, and should preferably have a short physical half-life so as to decrease the amount of radiation received by the body. The radioisotope used should preferably be pharmacologically inert, and, in the quantities administered, should not have any substantial physiological effect.

The ABM may be radio-labeled with different isotopes of iodine, for example ¹²³I, ¹²⁵I, or ¹³¹I (see for example, U.S. Pat. No. 4,609,725). The extent of radio-labeling must, however be monitored, since it will affect the calculations made based on the imaging results (i.e. a diiodinated ABM will result in twice the radiation count of a similar monoiodinated ABM over the same time frame).

In applications to human subjects, it may be desirable to use radioisotopes other than 1251 for labeling in order to decrease the total dosimetry exposure of the human body and to optimize the detectability of the labeled molecule (though this radioisotope can be used if circumstances require). Ready availability for clinical use is also a factor. Accordingly, for human applications, preferred radio-labels are for example, ^{99m}Tc, ⁶⁷Ga, ⁶⁸Ga, ⁹⁰Y, ¹¹¹In, ^{113m} In, ¹²³I, ¹⁸⁸ Re or ²¹¹At.

The radio-labeled ABM may be prepared by various methods. These include radio-halogenation by the chloramine-T method or the lactoperoxidase method and subsequent purification by HPLC (high pressure liquid chromatography), for example as described by J. Gutkowska et al in “Endocrinology and Metabolism Clinics of America: (1987) 16 (1):183. Other known methods of radio-labeling can be used, such as IODOBEADS™.

There are a number of different methods of delivering the radio-labeled ABM to the end-user. It may be administered by any means that enables the active agent to reach the agent's site of action in the body of a mammal. Because proteins are subject to being digested when administered orally, parenteral administration, i.e., intravenous, subcutaneous, intramuscular, would ordinarily be used to optimize absorption of an ABM, such as an antibody, which is a protein.

### EXAMPLES

Animal Models

Obesity and subsequent hyperinsulinemia and hyperglycemia were induced by feeding a group of 3 week old mice (50 C57B1/6 males) a high-fat diet (Bio-Serve, Frenchtown, N.J., #F1850 High Carbohydrate-High Fat). Another group of 3 week old mice (20 C57B1/6 males) were fed the normal control diet (PMI Nutrition International Inc., Brentwood, Mo., Prolab RMH3000). The mice were placed onto the respective diets immediately following weaning. Animal weights were determined weekly. Fasting blood-glucose and plasma insulin measurements were determined after 2, 4, 8 and 16 weeks, and 6 months, on the respective diets.

Normal weight, normal fasting blood glucose and normal fasting plasma insulin levels are defined as the respective mean values of the animals fed the control diet.

Two of the “most typical” animals were selected for each group (Control, hyperinsulinemic and Diabetic) at each time point ( 2,4, 8, and 16 weeks, and 6 months, after commencement of diet) for sacrifice. The selected mice were sacrificed and liver tissue obtained and frozen in liquid notrogen until processed for RNA isolation.

Fasting Blood Glucose Levels

Blood glucose levels was measured from a drop of blood taken from the tip of the tail of fasted (6 hr) mice using a Lifescan Genuine One Touch glucometer. All measurements occurred between 3:00 pm and 5:00 pm.

Plasma Insulin Measurements

Blood was collected from the tail of fasted (6hr) mice into a heparinized capillary tube and stored on ice. All collections occurred between 3:00 pm and 5:00 pm. Plasma was separated from red blood cells by centrifugation for 10 minutes at 8000 ×g and then stored at −20° C. Insulin concentrations were determined using the Rat Insulin ELISA kit and rat insulin standards (ALPCO) essentially as instructed by the manufacturer. Values were adjusted by a factor of 1.23 as determined by the manufacturer to correct for the species difference in cross-reactivity with the antibody.

RNA Isolation

Total RNA was isolated from livers using the RNA STAT-60 Total RNA/mRNA Isolation Reagent according to the manufacturer's instructions (Tel-Test, Priendswood, Tex.).

Sample Quantification and Quality Assessment

Total RNA was quantified and assessed for quality on a Bioanalyzer RNA 6000 Nano chip (Agilent). Each chip contained an interconnected set of gel-filled channels that allowed for molecular sieving of nucleic acids. Pin-non-overlapping and unrelated. Combinations of the above are also possible. In addition to the classes stated, the corresponding human gene clusters are also of interest. These may be obtained in a number of ways. First, one may search on Unigene (http://www.ncbi.nlm.nih.gov/entrez/query.fcgi?db=unigene) for the identified human protein. Review the “hits” (each of which is a Unigene record) for those prefixed by “Hs.” Secondly, one may access the Unigene record for the mouse gene cluster (which is given in Master Table 1), and then click on “Homologene”. This will bring up a new page which includes the section “Possible Homologous Genes”. One of the entries should be a Homo sapiens gene (considered by Unigene to be the most related human gene); click on its Unigene record link.

Additional information of interest may be accessed by searching with the mouse gene accession # in the Mouse Gene Informatics database, at http://www.informatics.jax.org/.

LENGTHY TABLE REFERENCED HERE
US20070142311A1-20070621-T00001
Please refer to the end of the specification for access instructions.

LENGTHY TABLE REFERENCED HERE
US20070142311A1-20070621-T00002
Please refer to the end of the specification for access instructions.

LENGTHY TABLE REFERENCED HERE
US20070142311A1-20070621-T00003
Please refer to the end of the specification for access instructions.

LENGTHY TABLE REFERENCED HERE
US20070142311A1-20070621-T00004
Please refer to the end of the specification for access instructions.

LENGTHY TABLE REFERENCED HERE
US20070142311A1-20070621-T00005
Please refer to the end of the specification for access instructions.

LENGTHY TABLE REFERENCED HERE
US20070142311A1-20070621-T00006
Please refer to the end of the specification for access instructions.

### REFERENCES

1. Unger, R. H., Foster, D. W. (1998) Diabetes mellitus. In Williams Textbook of Endocrinology, J. D. Wilson, D. W. Foster, H. M. Kronenberg, and P. R. Larsen, eds. (Philadelphia, W. B. Saunders Company), pp. 973-1059.
2. Polonsky, K. S. (1995) The beta-cell in diabetes: from molecular genetics to clinical research. Diabetes 44:705-717
3. Velho, G., Froguel, P. (1997) Genetic determinants of non-insulin-dependent diabetes mellitus: strategies and recent results. Diabete et Metabolisme 23:7-17
4. Groop, L. C., Tuomi, T. (1997) Non-insulin-dependent diabetes mellitus-a collision between thrifty genes and an affluent society. Ann. Med. 29:37-53.
5. Reaven, G. M. (1988) Role of insulin resistance in human disease. Diabetes 37:1595-1607.
6. Clark, M. G., Rattigan, S., Clark, D. G. (1983) Obesity with insulin resistance: experimental insights. Lancet (ii) 1236-1240.
7. Kissebah, A. H., Vydelingum, N., Murray, R., Evans, D. J., Hartz, A. J., Kakloff, R. K., Adams, P. W. (1982) Relation of body fat distribution to metabolic complications of obesity. J Clin. Endo and Metab 54(2):254-260.
8. Kissebah, A. H. (1996) Intra-abdominal fat: is it a major factor in developing diabetes and coronary artery disease? Diabetes Res Clin Pract 30 (Suppl):25-30.
9. Friedman, J. M., Leibel, R. (1992) Tackling a weighty problem. Cell 69:217-220
10. Bjorntorp, P. (1991) Metabolic implications of body fat distribution. Diabetes Care 14:1132-1143.
11. Emery, E. M., Schmid, T. L., Kahn, H. S., Filozof, P. P. (1993) A review of the association between abdominal fat distribution, health outcome measures, and modifiable risk factors. Am J Health Promot 7:342-353.
12. Wickelgren, I. (1998) Obesity: how big a problem? Science 280:1365.
13. Surwit, R. S., Kuhn, C. M., Cochrane, C., McCubbin, J. A., Feinglos, M. N. (1988) Diet-induced type-II diabetes in C57BL/6J mice. Diabetes 37:1163-11672.
14. Surwit, R. S., Feinglos, M. N., Rodin, J., Sutherland, A., Petro, A. E., Opara, E. C., Kuhn, C. M., Rebuffe-Scrive, M. (1995) Differential effects of fat and sucrose on the development of obesity and diabetes in C57BL/6J and A/J mice. Metabolism 44(5) :645-651.
15. Ahren, B. E., Simonson, E., Scheurink, A. J. W., Mulder, H., Myerson, U., Sundler, F. (1997) Dissociated insulinotropic sensitivity to glucose and carbachol in high-fat diet-induced insulin resistance in C57BL/6J mice. Metabolism 46(1):97-106. 16. Page, R., Morris, C., Williams, J., von Ruhland, C., Malik, A. N. (1997) Isolation of diabetes-associated kidney genes using differential display. Biochem Biophys Res Commun 232(1):49-53
17. Condorelli, G., Vigliotta, G., Iavarone, C., Caruso, M., Tocchetti, C. G., Andreozzi, F., Cafieri, A., Tecce, M. F., Formisano, P., Beguinot, L., Beguinot, F. (1998) PED/PEA-15 gene controls glucose transport and is overexpressed in type 2 diabetes mellitus. Embo J 17(14):3858-66
18. Peraldi, M. N., Berrou, J., Hagege, J., Rondeau, E., Sraer, J. D. (1998) Subtractive hybridization cloning: an efficient technique to detect overexpressed mRNAs in diabetic nephropathy. Kidney Int 53(4):926-31
19. Song, Y., Ailenberg, M., Silverman, M. (1998) Cloning of a novel gene in the human kidney homologous to rat munc13s: its potential role in diabetic nephropathy. Kidney Int 53(6):1689-95
20. Imagawa, M., Tsughiya, T., and Nishihara, T. (1999) Identification of inducible genes at the early stage of adipocyte differentiation of 3T3-L1 cells. Biochem. Biophys. Res. Comm. 254:299-305.
21. Nadler, S. T., Stoehr, J. P., Schueler, K. L., Tanimoto, G., Yandell, B. S., Attie, A. D. (2000) The expression of adipogenic genes is decreased in obesity and diabetes mellitus. Proc Natl Acad Sci USA 97:11371-11376

22. Lan H, Rabaglia M E, Stoehr J P, Nadler S T, Schueler K L, Zou F, Yandell B S, Attie A D. (2003) Gene expression profiles of nondiabetic and diabetic obese mice suggest a role of hepatic lipogenic capacity in diabetes susceptibility. Diabetes 52:688-700.

LENGTHY TABLE
The patent application contains a lengthy table section. A copy of the table is available in electronic form from the USPTO web site (http://seqdata.uspto.gov/?pageRequest=docDetail&DocID=US20070142311A1). An electronic copy of the table will also be available from the USPTO upon request and payment of the fee set forth in 37 CFR 1.19(b)(3).

1. A method of protecting a human subject from progression from a normoinsulinemic state to a hyperinsulinemic state, or from either to a type II diabetic state, which comprises administering to the subject a protective amount of at least one agent which is
(1) a polypeptide which is substantially structurally identical or conservatively identical in sequence to a reference protein which is (a) selected from the group consisting of mouse and human proteins set forth in master table 1, subtables 1A and 1C, or (b) selected from the group consisting of human proteins within at least one of the human protein classes set forth in master table 2, subtables 2A and 2C, or
(2) an expression vector encoding the polypeptide of (1) above and expressible in a human cell, under conditions conducive to expression of the polypeptide of (1);
where said agent protects said subject from progression from a normoinsulinemic state to a hyperinsulinemic state, or from either to a type II diabetic state.

2. A method of protecting a human subject from progression from a normoinsulinemic state to a hyperinsulinemic state, or from either to a type II diabetic state which comprises administering to the subject a protective amount of at least one agent which is
(1) an antagonist of a polypeptide, occurring in said subject, which is substantially structurally identical or conservatively identical in sequence to a reference protein which is (a) selected from the group consisting of mouse and human proteins set forth in master table 1, subtable 1B and 1C, or (b) selected from the group consisting of human proteins belonging to at least one of the human protein classes set forth in master table 2, subtables 2B and 2C,
(2) an anti-sense vector which inhibits expression of said polypeptide in said subject,
where said agent protects said subject from progression from a normoinsulinemic state to a hyperinsulinemic state, or from either to a type II diabetic state.

3. A method of screening for human subjects who are prone to progression from a normoinsulinemic state to a hyperinsulinemic state, or from either to a type II diabetic state, which comprises assaying tissue or body fluid samples from said subjects to determine the level of expression of at least one “favorable” human marker gene, said human marker gene encoding a human protein which is substantially structurally identical or conservatively identical in sequence to a reference protein which is (a) selected from the group consisting of mouse and human proteins set forth in master table 1, subtables 1A and 1C, or (b) selected from the group consisting of human proteins within at least one of the human protein classes set forth in master table 2, subtables 2A and 2C,
and directly correlating the level of expression of said marker gene with the propensity to progression in said patient.

4. A method of screening for human subjects who have a propensity for progression from a normoinsulinemic state to a hyperinsulinemic state, or from either to a type II diabetic state, which comprises assaying tissue or body fluid samples from said subjects to determine the level of expression of at least one “unfavorable” human marker gene, said human marker gene encoding a human protein which is substantially structurally identical or conservatively identical in sequence to a reference protein which is (a) selected from the group consisting of mouse and human proteins set forth in master table 1, subtable 1B and 1C, or (b) selected from the group consisting of human proteins belonging to at least one of the human protein classes set forth in master table 2, subtables 2B and 2C,
and inversely correlating the level of expression of said marker gene with the propensity to progression in said patient.

5. The method of claims 1 or 3 in which the reference protein is of subtable 1A or of a class set forth in subtable 2A.

6. The method of claims 1 or 3 in which the reference protein is of subtable 1B or of a class set forth in subtable 2B.

7. The method of any one of claims 1-6 in which (a) applies.

8. The method of any one of claims 1-7 in which the reference protein is a human protein.

9. The method of any one of claims 1-7 in which the reference protein is a mouse protein.

10. The method of any one of claims 3 or 4 in which the level of expression of the marker protein is ascertained by measuring the level of the corresponding messenger RNA.

11. The method of any one of claims 3 or 4 in which the level of expression is ascertained by measuring the level of a protein encoded by said marker gene.

12. The method of any one of claims 1-9 in which said polypeptide is at least 80% identical or at least highly conservatively identical to said reference protein.

13. The method of any one of claims 1-10 in which said polypeptide is at least 90% identical to said reference protein.

14. The method of any one of claims 1-11 in which said polypeptide is identical to said reference protein.

15. The method of any one of claims 1-14 in which the E-value cited for the reference protein in Master Table 1 is not more than e-6.

16. The method of claim 15 in which the E-value cited for the reference protein in Master Table 1 is less than e-10.

17. The method of claim 17 in which the E value calculated by BLASTN or BLASTX would be less than e-15, more preferably less than e-20, still more preferably less than e-40, even more preferably less than e-60, considerably more preferably less than e-80, and most preferably less than e-100.

18. The method of any of claims 2-17 in which the antagonist is an antibody, or an antigen-specific binding fragment of an antibody.

19. The method of any of claims 2-17 in which the antagonist is a peptide, peptoid, nucleic acid, or peptide nucleic acid oligomer.

20. The method of any of claims 2-17 in which the antagonist is an organic molecule with a molecular weight of less than 5,00 daltons.

21. The method of claim 20 in which said organic molecule is identifiable as a molecule which binds said polypeptide by screening a combinatorial library.
